(19) United States
(12) Patent Application Publication
(10) Pub. No.: US 2024/0159739 A1
(43) Pub. Date: May 16, 2024

(54) MEANS AND METHODS FOR ASSESSING IMMUNOTHERAPY

(71) Applicants: Deutsches Krebsforschungszentrum Stiftung des öffentlichen Rechts, Heidelberg (DE); Technische Universität München, München (DE)

(72) Inventors: Mathias HEIKENWAELDER, Heidelberg (DE); Percy KNOLLE, München (DE); Dominik PFISTER, Heidelberg (DE); Michael DUDEK, München (DE)

(21) Appl. No.: 18/552,028

(22) PCT Filed: Mar. 22, 2022
(86) PCT No.: PCT/EP2022/057450

§ 371 Date: Sep. 22, 2023

(30) Foreign Application Priority Data
Mar. 22, 2021 (EP) 21164116.2

(51) Int. Cl. G01N 33/50; G01N 33/68

(52) U.S. Cl. CPC G01N 33/505; G01N 33/6893; G01N 2333/70521; G01N 2800/52

(57) ABSTRACT

The present invention concerns the field of diagnostics and patient stratification for cancer therapy. In particular, it relates to a method for assessing a treatment response associated with immunotherapy in a subject in need thereof comprising the steps of determining hepatic auto-aggressive CD8 positive (+) PD-1 positive (+) T cells exhibiting traits of activation and exhaustion or CD8+ T cell precursors thereof in a sample of a subject in need of immunotherapy or in a data set comprising imaging data of a subject in need of immunotherapy, and assessing the treatment response associated with immunotherapy based on the presence, absence or abundance of said hepatic auto-aggressive CD8+ PD-1+ T-cells exhibiting traits of activation or exhaustion or CD8+ T cell precursors thereof. Further contemplated is a method for recommending immunotherapy for a subject or a method for treating a subject by immunotherapy. The present invention also provides a diagnostic device for carrying out the method of the present invention.

The present invention concerns the field of diagnostics and patient stratification for cancer therapy. In particular, it relates to a method for assessing a treatment response associated with immunotherapy in a subject in need thereof comprising the steps of determining hepatic, auto-aggressive CD8 positive (+) PD-1 positive (+) T cells exhibiting traits of activation and exhaustion or hepatic CD8+ T cell precursors thereof sin a sample of a subject in need of immunotherapy or in a data set comprising imaging data of a subject in need of immunotherapy, and assessing the treatment response associated with immunotherapy based on the presence, absence or abundance of said hepatic CD8+ PD-1+ T-cells exhibiting traits of activation or exhaustion or hepatic CD8+ T cell precursors thereof. Further contemplated is a method for recommending immunotherapy for a subject or a method for treating a subject by immunotherapy. The present invention also provides a diagnostic device for carrying out the method of the present invention.

Potentially curative hepatocellular carcinoma (HCC)-treatment options such as liver-transplantation, tumor-resection, or ablation are limited to early-stage tumors (Llovet 2021; Galle 2018). Multikinase-inhibitors or anti-VEGF-R2 antibodies have been approved for advanced HCC immunotherapy that is considered to activate T cells or reinvigorate immune-surveillance against cancer showed response rates of 21-26% (Duffy 2016, Sangro 2013).

Nivolumab and pembrolizumab (PD1-directed antibodies) were approved for HCC treatment Zhu 2018, El-Khoueiry 2017), although phase III trials failed to reach primary endpoints to increase survival. Combination of atezolizumab (anti-PD-L1)/bevacizumab (anti-VEGF) demonstrated increased overall/progression-free survival in a phase III trial, making it the first-line treatment for advanced HCC (Finn 2020).

One issue affecting immunotherapy efficacy may be the impact of different underlying HCC-etiologies, with distinct hepatic environments driving HCC-induction and local regulation of immune responses by distinct mechanisms (Roderburg 2020).

Nonalcoholic fatty liver disease (NAFLD) is an HCC-etiology with pandemic dimension, affecting >200 million people worldwide (Anstee 2019) with a trend in strongly rising further. Approximately 10-20% of individuals with NAFLD progress over time from steatosis to NASH. Innate and adaptive immune-cell activation in combination with increased metabolites and endoplasmic reticulum stress (Wolf 2014, Ma 2016, Malehmir 2019, Nakagawa 2014) are believed to lead to a cycle of hepatic necro-inflammation and regeneration that potentially leads to HCC (Ringelhan 2018, Michelotti 2013, Friedman 2018). NASH has become an emerging HCC-risk factor.

Accordingly, there is a need for stratifying patients suffering from HCC without known etiology with respect to their benefit from immunotherapy.

The technical problem underlying the present invention may be seen as the provision of means and methods for complying with the aforementioned needs. The technical problem is solved by the embodiments characterized in the claims and herein below.

The present invention, thus, relates to a method for assessing a treatment response associated with immunotherapy in a subject in need thereof comprising the steps of:
(a) determining (i) hepatic auto-aggressive CD8 positive (+) PD-1 positive (+) T cells exhibiting traits of activation and exhaustion or (ii) hepatic CD8+ T cell precursors thereof in a sample of a subject in need of immunotherapy; and
(b) assessing the treatment response associated with immunotherapy based on the presence, absence or abundance of (i) said hepatic auto-aggressive CD8+ PD-1+ T-cells exhibiting traits of activation and exhaustion or (ii) said hepatic CD8+ T cell precursors thereof.

It is to be understood that as used in the specification and in the claims, “a” or “an” can mean one or more, depending upon the context in which it is used. Thus, for example, reference to “an” item can mean that at least one item can be utilized.

As used in the following, the terms “have”, “comprise” or “include” or any grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements. The term “comprising” also encompasses embodiments where only the items referred to are present, i.e. it has a limiting meaning in the sense of “consisting of” or “essentially consisting of”.

Further, as used in the following, the terms “preferably”, “more preferably”, “most preferably”, “particularly”, “more particularly”, “typically”, and “more typically” or similar terms merely specify preferred subject matter but shall not be meant to limit generic subject matter. Thus, features introduced by these terms are not intended to restrict the scope of the claims in any way. The invention may, as the skilled person will recognize, be performed by not using the introduced features or by using alternative features.

Further, it will be understood that the term “at least one” as used herein means that one or more of the items referred to following the term may be used in accordance with the invention. For example, if the term indicates that at least one biomarker shall be used this may be understood as one biomarker or more than one biomarker, i.e. two, three, four, five or any other number. Depending on the item the term refers to the skilled person understands as to what upper limit the term may refer, if any.

The method of the present invention may consist of the aforementioned step or may comprise additional steps, such as steps for further evaluation of the assessment obtained in step (b), steps recommending therapeutic measures such as treatments, or the like. Moreover, it may comprise steps prior to step (a) such as steps relating to sample pre-treatments. However, preferably, it is envisaged that the above-mentioned method is an ex vivo method which does not require any steps being practiced on the human or animal body. Moreover, the method be assisted by automation. Typically, the determination of the cells may be supported by robotic equipment while the assessment may be supported by data processing equipment such as computers. Further details are to be found elsewhere herein.

The diseases and disorders referred to in accordance with the present invention are well known in the art and the clinical signs and symptoms associated therewith are described in standard text books of medicine such as Stedman's Medical Dictionary or the like. “Liver cancer” as referred to herein relates, typically, to hepatocellular carcinoma or cholangiocarcinoma. The term “non-liver cancer” relates to cancer entities which do not affect the liver. Preferably, non-cancer entities according to the present invention are melanoma, prostate cancer, colon cancer or breast cancer. Non-alcoholic fatty liver disease is a well-known medical condition characterized by extra fat in the liver which is not caused by excessive alcohol. Typically, extra fat of more than 5% of the liver weight is indicative for fatty liver. Nonalcoholic fatty liver disease may develop into nonalcoholic steatohepatitis including liver swelling and inflammation. Said Nonalcoholic steatohepatis may cause liver fibrosis, cirrhosis, and, as a final stage, liver cancer. The term “systemic obesity” or “metabolic syndrome” as used herein refers to a disorder comprising a cluster of three or more of the following conditions: central obesity, high blood pressure, high blood sugar, high serum triglycerides and low serum high density lipoprotein (HDL).

The term “immunotherapy” as referred to in accordance with the present invention encompasses mono-immunotherapies, i.e. immunotherapies involving administration of one immunotherapeutic drug, as well as combination immunotherapies, i.e. immunotherapies involving administration of more than one immunotherapeutic drugs. Preferably, immunotherapy according to the present invention involves PD-1 and/or PD-L1 targeted immunotherapy. Antibody therapy against PD-1 may encompass administering of drugs such as nivolumab or pembrolizumab. Also envisaged as immunotherapy according to the invention is a combination therapy using anti-PD-L1 (e.g., atezolizumab) and anti-VEGF (e.g., bevacizumab). Moreover, the immunotherapy may also encompass administration of further anti-cancer drugs or drugs that support the therapy.

The term “treatment response” as used herein refers to any biological response of that occurs in a subject which is treated by immunotherapy. Said response may be a therapeutically effective response involving amelioration or cure of the disease or disorder to be treated by the immunotherapy or amelioration or cure of symptoms accompanying it. Preferably, said therapeutically effective treatment response refers to cases where the subject benefits from the immunotherapy. More preferably, a therapeutically effective treatment according to the invention comprises amelioration or cure of liver cancer, preferably, hepatocellular carcinoma (HCC) or cholangiocarcinoma (CCA). A therapeutically effective response is, typically, observed in accordance with the present invention when immunotherapy is to be used for the treatment or prevention of liver cancer, preferably HCC or CCA. Moreover, it may also include any adverse response to an immunotherapy such as a response that comprises the progression or persistence of liver cancer, preferably, HCC or CCA or intrahepatic metastasis of any origin. Typically, an adverse treatment response observed in accordance with the present invention when immunotherapy is to be used for the treatment or prevention of liver cancer comprises the progression or persistence of liver cancer, preferably, HCC or CCA or intrahepatic metastasis of any origin, or liver damage. Moreover, a treatment response as referred to in accordance with the present invention includes a non-response, i.e. a case where no clinically relevant physiological changes in the status of the subject are observable. Yet, a therapeutic response upon administration of immunotherapy may also involve the development of a hepatic adverse side effect. Preferably, said hepatic adverse side effect occurs in cases where the subject is not in need of immunotherapy for the treatment of liver cancer but suffers from other cancer entities. More preferably, the subject suffers from non-liver cancer susceptible to systemic immunotherapy, preferably, melanoma, prostate cancer, colon cancer or breast cancer. More preferably, the subject in such a case involving development of a hepatic adverse side effect suffers or is suspected to suffer from nonalcoholic fatty liver disease (NAFLD) or systemic obesity (metabolic syndrome). Said adverse hepatic side effect, preferably, comprises development of liver damage, liver dysfunction or liver cancer, preferably, HCC or CCA.

The term “assessing” as used herein refers to determining a treatment response (i.e. an adverse response, a non-response, a therapeutically effective response or an adverse hepatic side effect) of the subject to immunotherapy. This includes determining said treatment response in the subjects current physiological state in a diagnostic approach. Moreover, the term also encompasses determining whether a subject will develop a treatment response in accordance with the present invention (i.e. an adverse response, a non-response, a therapeutically effective response or an adverse hepatic side effect) in the future, i.e. within a certain predictive window, in a prognostic approach. Thus, the assessment also allows for stratifying subjects with respect to being susceptible to immunotherapy, or not. Moreover, assessing may also include approaches where a subject is monitored for a treatment response over time, e.g., in case the immunotherapy is administered over a certain period of time as a therapeutic or preventive measure. As will be understood by those skilled in the art, such an assessment, although preferred to be, may usually not be correct for 100% of the investigated subjects. The term, however, requires that a statistically significant portion of subjects can be correctly assessed.

Whether a portion is statistically significant can be determined without further ado by the person skilled in the art using various well known statistic evaluation tools, e.g., determination of confidence intervals, p-value determination, Student's t-test, Mann-Whitney test, etc. Details may be found in Dowdy and Wearden, Statistics for Research, John Wiley & Sons, New York 1983. Typically envisaged confidence intervals are at least 50%, at least 60%, at least 70%, at least 80%, at least 90%, at least 95%. The p-values are, typically, 0.2, 0.1, 0.05.

The term “sample” as used herein refers to any biological sample material that comprises or is suspected to comprise hepatic auto-aggressive CD8 positive (+) PD-1 positive (+) T cells exhibiting traits of activation and exhaustion or hepatic CD8+ T cell precursors thereof. Preferably, the sample is a tissue, cell or fluid sample obtainable from the liver, more preferably, the sample is a liver biopsy sample, most preferably, comprising liver tissue.

The term “subject” as used herein refers to a mammal, preferably, a human, pet, farming animal or laboratory animal such as a rodent, preferably, a mouse or rat. More preferably, said subject is a human. The subject shall be in need of an immunotherapy. This encompasses subjects which are in need of immunotherapy due to apparent diseases or disorders that are susceptible to treatment by immunotherapy, such as viral-related or non-viral-related liver cancer, melanoma, prostate cancer, colon cancer, cervix cancer or breast cancer. Moreover, a subject in need of immunotherapy may also be a subject suspected to be susceptible or to benefit from administration of immunotherapy. Preferably, this includes a subject which may receive immunotherapy after successful therapy of a disease or disorder in prevention of reoccurrence of the disease or disorder or a subject which receives immunotherapy as a preventive measure.

The term “hepatic auto-aggressive CD8 positive (+) PD-1 positive (+) T cells exhibiting traits of activation and exhaustion” as used herein refers to a population of T cells which is characterized by cell surface biomarker CD8 and PD-1, i.e. T cells being positive for CD8 and PD-1. Moreover, said CD8+ PD-1+ T cells shall exhibit traits of activation and exhaustion as characterized by the expression of biomarkers indicative for said traits. Furthermore, this type of CD+ PD-1+ T cells has been found to be hepatic auto-aggressive, i.e. auto-aggressive against liver tissue upon stimulation, e.g., by anti-PD-1 antibodies or PD-1L used in immunotherapy. As a result of this hepatic auto-aggressive behavior, T cells of said type will cause severe damage in the liver of subjects receiving systemic immunotherapy. Said severe damage caused includes the adverse treatment responses and adverse hepatic side effects described elsewhere herein. Since said treatment responses affect the liver, the CD8+ PD-1+ T cells must occur in the liver and, thus, must be determined as hepatic CD8+ PD-1+ T cells. Moreover, in addition to the liver, said T cells may also occur in the peripheral blood and are capable of entering the liver via the blood stream. It will be understood that the auto-aggressive T cells may also be auto-aggressive against tissues other than liver tissue. Thus, the hepatic auto-aggressive CD8+ PD-1+ T cells according to the present invention are, preferably, resident in the liver and/or present in the peripheral blood. Moreover, the auto-aggressive T cells must exhibit traits of activation and exhaustion. These traits are accompanied by, e.g., an increased expression compared to control CD8+ T cells of at least one biomarkers selected from the group consisting of: TOX, CXCR6, TNFα, LAG3, GZMB (Granzyme B) and TIGIT, more preferably, CXCR6 and TOX or a reduced expression compared to control CD8+ T cells of at least one biomarker selected from the group consisting of: KLF2, IL-7R, TCF7, Foxo1 and SELL. Thus, preferably, said hepatic auto-aggressive CD8+ PD-1+ T cells exhibiting traits of activation and exhaustion exhibit an increased expression compared to control CD8+ T cells of at least one biomarkers selected from the group consisting of: TOX, CXCR6, TNFα, LAG3, GZMB (Granzyme B) and

TIGIT, more preferably, CXCR6 and TOX. Also preferably, said hepatic auto-aggressive CD8+ PD-1+ T cells exhibiting traits of activation and exhaustion exhibit a reduced expression compared to control CD8+ T cells of at least one biomarker selected from the group consisting of: KLF2, IL-7R, TCF7, Foxo1 and SELL. More preferably, said hepatic auto-aggressive CD8+ PD-1+ T cells exhibiting traits of activation and exhaustion exhibit an increased expression compared to control CD8+ T cells of all biomarkers selected from the group consisting of: TOX, CXCR6, TNFα, LAG3, GZMB (Granzyme B) and TIGIT and, also preferably, exhibit a reduced expression compared to control CD8+ T cells of all biomarkers selected from the group consisting of: KLF2, IL-7R, TCF7, Foxo1 and SELL.

The term “CD8+ T cell precursors” as used herein refers to CD8+ T cells which undergo further, preferably irrevocably, differentiation into the hepatic auto-aggressive CD8 positive (+) PD-1 positive (+) T cells exhibiting traits of activation and exhaustion. Typically, such cells are programmed irrevocably already to become such cells. Preferably, said precursors are resident in the liver, i.e. are hepatic T cells, or are present in the peripheral blood, i.e. are peripheral blood derived T cells. Said CD8+ T cell precursors express already typical biomarkers such as TCF7, SELL and/or IL-7R. However, they can be further characterized by a change over time in the expression profile of further biomarkers, such as TOX, CXCR6, TNFα, LAG3, GZMB (Granzyme B) TIGIT, KLF2, IL-7R, TCF7, Foxo1 and SELL. The change in expression over time can be typically be observed within a time window of one month, two months, three months, six months, twelve months or 24 months. Thus, preferably, said CD8+ T cell precursors are characterized by at least one biomarker or all biomarkers selected from the group consisting of: TCF7, SELL, and IL-7R. Preferably, said CD8+ T cell precursors exhibit a change in expression over time of at least one biomarker or all of the biomarkers selected from the group consisting of: TOX, CXCR6, TNFα, LAG3, GZMB (Granzyme B) TIGIT, KLF2, IL-7R, TCF7, Foxo1 and SELL. More preferably, (i) said change is a decrease in expression over time if said at least one biomarker is selected from the group consisting of KLF2, IL-7R, TCF7, Foxo1 and SELL; and (ii) said change is an increase in expression over time if said at least one biomarker is selected from the group consisting of TOX, CXCR6, TNFα, LAG3, GZMB (Granzyme B) and TIGIT.

The presence, absence or abundance of said hepatic auto-aggressive CD8 positive (+) PD-1 positive (+) T cells exhibiting traits of activation and exhaustion or the CD8+ T cell precursors thereof can be determined in a sample, such as an organ, tissue or cell sample obtained by biopsy or from peripheral blood samples, by standard techniques of molecular biology including any kind of antibody- or aptamer-based detection of the biomarker proteins. Alternatively, transcripts encoding the biomarker proteins can be determined by suitable nucleic acid detection techniques. The skilled artisan is well aware of how biomarker proteins or transcripts encoding them can be determined qualitatively and quantitatively. Particular preferred techniques include immunohistochemical and histochemical analysis, in situ hybridization techniques, immunoassays, such as ELISA or RIA, Cell sorting such as FACS analysis, high-throughput RNA sequencing, single cell RNA sequencing analysis, RNA velocity analysis, mass spectroscopy, mass cytometry, MRI, and the like. Moreover, particular preferred techniques are described in the accompanying Examples below in detail.

More typically, determining the presence, absence or abundance of a biomarker referred to in accordance with the present invention comprises contacting the sample with a detection agent that specifically binds to the biomarker or a transcript encoding it. If protein or peptide biomarkers are to be detected by the detection agent, typically, an antibody, aptamer, peptide or protein may be used as a detection agent in accordance with the present invention. If a transcript encoding a biomarker protein is to be detected, it will be understood that, typically, a nucleic acid probe being either RNA or DNA may be used for detection as detecting agent according to the invention. Preferred detection agents are also described elsewhere herein in more detail. More typically, said detection agent can be identified upon binding by an intrinsic detectable label or may be bound by a molecule being or comprising such a label The detectable label in accordance with the present invention may by any compound or element that is capable of generating a detectable signal being associated with the biomarker upon binding. Typically, a detectable label may be a fluorescent molecule or moiety, a radioactive element, an enzyme, a chemoluminescent molecule or moiety, an electrochemically detectable molecule or moiety, an immunological tag, a mass tag, and the like. It will be understood that the label may be also comprised by a second detection molecule that specifically binds to the detection agent once bound to the biomarker, such as a secondary antibody or aptamer comprising a label. Preferred labels are described elsewhere herein in more detail.

An antibody as a detection agent as referred to herein encompass to all types of antibodies which specifically bind to the biomarker protein. Preferably, the antibody of the present invention is a monoclonal antibody, a polyclonal antibody, a single chain antibody, a chimeric antibody or any fragment or derivative of such antibodies being still capable of binding to the biomarker protein specifically. Such fragments and derivatives comprised by the term antibody as used herein encompass a bispecific antibody, a synthetic antibody, a Fab, F(ab)2 Fv or scFv fragment, or a chemically modified derivative of any of these antibodies. Specific binding as used in the context of the anti-body of the present invention means that the antibody does not cross react with other molecules present in the sample to be investigated. Specific binding can be tested by various well-known techniques. Antibodies or fragments thereof, in general, can be obtained by using methods which are described, e.g., in Harlow and Lane “Antibodies, A Laboratory Manual”, CSH Press, Cold Spring Harbor, 1988. Monoclonal antibodies can be prepared by the techniques which comprise the fusion of mouse myeloma cells to spleen cells derived from immunized mammals and, preferably, immunized mice. Preferably, an immunogenic peptide is applied to a mammal. The said peptide is, preferably, conjugated to a carrier protein, such as bovine serum albumin, thyroglobulin, and keyhole limpet hemocyanin (KLH). Depending on the host species, various adjuvants can be used to increase the immunological response. Such adjuvants encompass, preferably, Freund's adjuvant, mineral gels, e.g., aluminum hydroxide, and surface-active substances, e.g., lysolecithin, pluronic polyols, polyanions, peptides, oil emulsions, keyhole limpet hemocyanin, and dinitrophenol. Mono-clonal antibodies which specifically bind to an analyte can be subsequently prepared using the well-known hybridoma technique, the human B cell hybridoma technique, and the EBV hybridoma technique. Detection systems using antibodies are based on the highly specific binding affinity of antibodies for a specific antigen, i.e. the biomarker protein. Binding events result in a physicochemical change that can be detected as described elsewhere herein.

An aptamer as a detection agent according to the invention encompasses oligonucleic acid or peptide molecules that bind to a specific biomarker protein. Oligonucleic acid aptamers are engineered through repeated rounds of selection or the so-called systematic evolution of ligands by exponential enrichment (SELEX technology). Peptide aptamers comprise of a variable peptide loop attached at both ends to a protein scaffold. This double structural constraint shall increase the binding affinity of the peptide aptamer into the nano-molar range. Said variable peptide loop length is, typically, composed of ten to twenty amino acids, and the scaffold may be any protein having improved solubility and capacity properties, such as thioredoxin-A. Peptide aptamer selection can be made using different systems including, e.g., the yeast two-hybrid system. Detection systems using aptamers as detection agents can be based on specific antibody mimicking aptamers as aptasensors.

A nucleic acid molecule useful as a detection agent in accordance with the present invention refers to DNA or RNA molecules that are capable of specifically interacting with the transcript for the biomarker. The biomarker transcript is a nucleic acid molecule, too, and specific binding can be achieved via the specific interactions of complementary or reverse complementary nucleotide strands. Typically, the nucleic acid useful as detection agent is selected from the group consisting of: an antisense RNA, a ribozyme, a siRNA or a micro RNA. Also preferably, oligonucleotides having complementary and reverse complementary sequences may be used as target transcript specific primers for PCR-based detection techniques.

An antisense RNA as used herein refers to RNA which comprise a nucleic acid sequence which is essentially or perfectly complementary to the target transcript. Typically, an antisense nucleic acid molecule essentially consists of a nucleic acid sequence being complementary to at least 100 contiguous nucleotides, more preferably, at least 200, at least 300, at least 400 or at least 500 contiguous nucleotides of the target transcript. How to generate and use antisense nucleic acid molecules is well known in the art.

A ribozyme as used herein refers to catalytic RNA molecules possessing a well-defined tertiary structure that allows for specific binding to target RNA and catalyzing either the hydrolysis of one of their own phosphodiester bonds (self-cleaving ribozymes), or the hydrolysis of bonds in target RNAs, but they have also been found to catalyze the aminotransferase activity of the ribosome. How to generate and use such ribozymes is well known in the art.

A siRNA as used herein refers to small interfering RNAs (siRNAs) which are complementary to target RNAs (encoding a gene of interest) and diminish or abolish gene expression by RNA interference (RNAi). RNAi is generally used to silence expression of a gene of interest by targeting mRNA. Briefly, the process of RNAi in the cell is initiated by double stranded RNAs (dsRNAs) which are cleaved by a ribonuclease, thus producing siRNA duplexes. The siRNA binds to another intracellular enzyme complex which is thereby activated to target whatever mRNA molecules are homologous (or complementary) to the siRNA sequence. The function of the complex is to target the homologous mRNA molecule through base pairing interactions between one of the siRNA strands and the target mRNA. Thus, siRNA molecules are capable of specific binding and can be used as detection agents according to the present invention.

microRNA as used herein refers to a self-complementary single-stranded RNA which comprises a sense and an antisense strand linked via a hairpin structure. The micro RNA comprise a strand which is complementary to an RNA targeting sequences comprised by a transcript to be downregulated. micro RNAs are processed into smaller single stranded RNAs and, therefore, presumably also act via the RNAi mechanisms. How to design and to synthesize microRNAs which specifically bind and degrade a transcript of interest is known in the art and described, e.g., in EP 1 504 126 A2 in detail. Due to the specific nucleic acid binding capabilities, they can be used as detection agents according to the invention.

Detection systems using nucleic acid as detection molecules can be based on complementary base pairing interactions. The recognition process is based on the principle of complementary nucleic acid base pairing. If the target nucleic acid sequence is known, complementary sequences can be synthesized and labeled for detection. The hybridization event can be, e.g., detected. Moreover, using PCR-based techniques, even low amounts of transcripts can be determined and quantified. How to carry out such PCR-based techniques is well known to the skilled artisan.

A protein to be used as a detection agent in accordance with the present invention may be a protein that specifically interacts with the biomarker protein to be determined. Accordingly, depending on the nature of the said biomarker protein, the protein may be, typically, an enzyme, a receptor, a ligand, a signaling protein, a transcription factor or a structural protein. Moreover, parts of such proteins may be used as peptide detection molecules in accordance with the present invention, e.g., ligand binding domains or substrate binding pockets. Proteins in accordance with the present invention usually comprise at least 100 amino acids covalently linked by peptide bonds. They may further comprise modifications such as glycosylations, phosphorylation, methylation, ubiquitinylation or myristylations.

For example, the specific binding capabilities and catalytic activity of enzymes make them particular suitable as detection agents. Biomarker substrate recognition is enabled through several possible mechanisms: the enzyme converting the biomarker protein substrate into a product that is detectable, the detection of enzyme inhibition or activation by the biomarker protein or monitoring modification of enzyme properties resulting from interaction with the biomarker protein. Alternatively, receptor molecules may exhibit specific binding properties for their ligands and can be used as detection agents similar to antibodies.

A peptide suitable as detection agent according to the invention may be functional fragments of proteins which are still capable of specifically binding to the biomarker protein to be detected. Thus, peptides useful as detection molecules may ligand binding domains or substrate binding pockets. Moreover, peptides may be artificially generated and selected for specific binding to the biomarker protein by screening artificial peptide libraries. Peptides typically comprise less than 100 amino acids which are linked by covalent peptide bonds. Peptides may be modified as well. For example, small protein scaffolds with favorable biophysical properties have been engineered to generate artificial binding peptides. These peptide molecules are capable of specific binding to different biomarker proteins. Typically, these artificial binding proteins are much smaller than antibodies (usually less than 100 amino-acid residues), have a strong stability, lack disulfide bonds and can be expressed in high yield in reducing cellular environments like the bacterial cytoplasm, contrary to antibodies and their derivatives.

Typical labels which may be used in accordance with the invention include gold particles, latex beads, acridan ester, luminol, ruthenium, enzymatically active labels, radioactive labels, magnetic labels, e.g., magnetic beads, including paramagnetic and superparamagnetic labels, and fluorescent labels. Enzymatically active labels include e.g. horseradish peroxidase, alkaline phosphatase, beta-Galactosidase, Luciferase, and derivatives thereof. Suitable substrates for detection include di-amino-benzidine (DAB), 3,3′-5,5′-tetramethylbenzidine, NBT-BCIP (4-nitro blue tetrazolium chloride and 5-bromo-4-chloro-3-indolyl-phosphate. A suitable enzyme-substrate combination may result in a colored reaction product, fluorescence or chemoluminescence, which can be measured according to methods known in the art (e.g. using a light-sensitive film or a suitable camera system). As for measuring the enzymatic reaction, the criteria given above apply analogously. Typical fluorescent labels include fluorescent proteins (such as GFP and its derivatives), Cy3, Cy5, Texas Red, Fluorescein, and the Alexa dyes. Also the use of quantum dots as fluorescent labels is contemplated. Typical radioactive labels include 35S, 125I, 32P, 33P and the like. A radioactive label can be detected by any method known and appropriate, e.g. a light-sensitive film or a phosphor imager. Suitable labels may also be or comprise tags, such as biotin, digoxygenin, His-, GST-, FLAG-, GFP-, MYC-tag, influenza A virus haemagluttinin (HA), maltose binding protein, and the like.

The term “biomarker” as used in accordance with the present invention relates to a molecule which is expressed by the hepatic auto-aggressive CD8 positive (+) PD-1 positive (+) T cells exhibiting traits of activation and exhaustion or the CD8+ T cell precursors thereof. Accordingly, the biomarker protein and/or its transcript may serve as a biomarker in accordance with the invention. A transcript used as biomarker in accordance with the present invention may be typically an mRNA or precursor thereof. The biomarker according to the invention is, preferably, a molecule which is indicative for a certain physiological or pathological status of a cell. However, it must not necessarily be the cause of or be in any causal relationship to said status.

The term “CD8” as used herein refers to “cluster of differentiation 8” which is a transmembrane glycoprotein being a member of the immunoglobulin superfamily with an immunoglobulin variable (IgV)-like extracellular domain connected to the membrane that serves as a co-receptor for the T-cell receptor (TCR). Along with the TCR, the CD8 co-receptor plays a role in T cell signaling and cytotoxic T cell antigen interactions. Two isoforms of the protein are known, CD8 alpha and CD8 beta. CD8 forms a dimer, consisting of a pair of CD8 chains. The most common form of CD8 is composed of a CD8 alpha and CD8 beta chain. Preferably, human CD8 alpha has an amino acid sequence as deposited in the Uniprot database under accession number P01732, human CD8 beta has an amino acid sequence as deposited in the Uniprot database under accession number P10966. Preferably, mouse CD8 alpha has an amino acid sequence as deposited in the Uniprot database under accession number P01731, mouse CD8 beta has an amino acid sequence as deposited in the Uniprot database under accession number P10300. The term also encompasses variants of the aforementioned CD8 proteins.

Variants of biomarker proteins as referred to herein shall have at least the same essential biological and immunological properties as the aforementioned biomarker protein. In particular, they share the same essential biological and immunological properties if they are detectable by the same specific assays referred to in this specification, e.g., by ELISA assays using polyclonal or monoclonal antibodies specifically recognizing the biomarker protein. Moreover, it is to be understood that a variant as referred to in accordance with the present invention shall have an amino acid sequence which differs due to at least one amino acid substitution, deletion and/or addition wherein the amino acid sequence of the variant is still, preferably, at least 50%, 60%, 70%, 80%, 85%, 90%, 92%, 95%, 97%, 98%, or 99% identical with the amino sequence of the specific biomarker protein, preferably over the entire length thereof. The degree of identity between two amino acid sequences can be determined by algorithms well known in the art. Preferably, the degree of identity is to be determined by comparing two optimally aligned sequences over a comparison window, where the fragment of amino acid sequence in the comparison window may comprise additions or deletions (e.g., gaps or overhangs) as compared to the reference sequence (which does not comprise additions or deletions) for optimal alignment. The percentage is calculated by determining the number of positions at which the identical amino acid residue occurs in both sequences to yield the number of matched positions, dividing the number of matched positions by the total number of positions in the window of comparison and multiplying the result by 100 to yield the percentage of sequence identity. Optimal alignment of sequences for comparison may be carried out by comparison algorithms well known in the art. Preferably, algorithms implemented in GAP, BESTFIT, BLAST, FAST, PASTA, and TFASTA (Wisconsin Genetics Software Package, Genetics Computer Group (GCG), 575 Science Dr., Madison, WI) may be used. Preferably, the default values of 5.00 for gap weight and 0.30 for gap weight length are used. Variants referred to above may be allelic variants or any other species specific homologs, paralogs, or orthologs.

The term “PD-1” as used herein refers to “Programmed cell death protein 1”, also known as CD279 (“cluster of differentiation 279”). PD-1 is a surface receptor protein of the immunoglobulin superfamily that plays a role in regulating immune response by down-regulating the immune system and promoting self-tolerance by suppressing T cell inflammatory activity. It, thus, prevents autoimmune diseases, but it can also prevent the immune system from killing cancer cells. PD-1 acts through two mechanisms. First, it promotes apoptosis of antigen-specific T-cells; second, it reduces apoptosis in regulatory T cells. The PD-1 protein in humans is encoded by the PDCD1 gene. PD-1 is typically expressed on T cells and pro-B cells and binds to two ligands, PD-L1 and PD-L2. Preferably, human PD-1 has an amino acid sequence as deposited in the Uniprot database under accession number Q15116, mouse PD-1 has an amino acid sequence as deposited in the Uniprot database under accession number Q02242. The term also encompasses variants of the aforementioned PD-1 proteins.

The term “TOX” as used herein refers to “thymocyte selection-associated high mobility group box protein”. TOX is a protein of the large superfamily of chromatin associated proteins that share an approximately 75 amino acid DNA binding motif, the HMG (high mobility group)-box. While TOX has a single HMG-box motif, it is predicted to bind DNA in a sequence-independent manner. TOX is also a member of a small subfamily of proteins (TOX2, TOX3, and TOX4) that share almost identical HMG-box sequences and are suggested to be involved in tumor formation. TOX is highly expressed in the thymus, the site of development of T cells. TOX is necessary for T cell persistence but also drives T-cell “exhaustion” thus contributing to diminished anti-tumor or anti-viral function in these cells.

The term “CXCR6” as used herein refers to “C-X-C chemokine receptor type 6” a seven transmembrane receptor like protein. It is a chemokine receptor present on the surface of lymphocytes and being involved in inflammation processes and viral infection processes. Its ligand is the cytokine CXCL16. Preferably, human CXCR6 has an amino acid sequence as deposited in the Uniprot database under accession number O00574, mouse CXCR6 has an amino acid sequence as deposited in the Uniprot database under accession number Q9EQ16. The term also encompasses variants of the aforementioned CXCR6 proteins.

The term “TNFα” as used herein refers to “Tumor necrosis factor alpha” which is a cytokine released during inflammation processes. TNF is a member of the TNF superfamily, which consists of various transmembrane proteins with a homologous TNF domain. TNF signaling occurs through two receptors: TNFR1 and TNFR2. TNFR1 is almost ubiquitously expressed, while TNRF2 is restricted primarily to endothelial, epithelial, and subsets of immune cells. TNFR1 signaling tends to be pro-inflammatory and apoptotic, whereas TNFR2 signaling is anti-inflammatory and promotes cell proliferation. Suppression of TNFR1 signaling has been important for treatment of autoimmune disease, whereas TNFR2 signaling promotes wound healing. TNF-α exists as a transmembrane form (mTNF-α) and as a soluble form (sTNF-α) both of which are encompassed in accordance with the present invention. sTNF-α results from enzymatic cleavage of mTNF-α. Preferably, human TNFα has an amino acid sequence as deposited in the Uniprot database under accession number P01375, mouse TNFα has an amino acid sequence as deposited in the Uniprot database under accession number P06804. The term also encompasses variants of the aforementioned TNFα proteins.

The term “LAG3” as used herein refers to “Lymphocyte-activation gene 3” a cell surface receptor with diverse biologic effects on T cell function. The LAG3 protein, which belongs to immunoglobulin (Ig) superfamily, comprises a 503-amino acid type I transmembrane protein with four extracellular Ig-like domains, designated D1 to D4. Preferably, human LAG3 has an amino acid sequence as deposited in the Uniprot database under accession number P18627, mouse LAG3 has an amino acid sequence as deposited in the Uniprot database under accession number Q61790. The term also encompasses variants of the aforementioned LAG3 proteins.

The term “GZMB” or “granzyme B” as used herein refers to a serine protease (E.C. 3.4.21.79) typically found in the granules of natural killer cells (NK cells) and cytotoxic T cells. It is secreted by these cells along with the pore forming protein perforin to mediate apoptosis in target cells. It has also various secondary functions including functions in inducing inflammation by stimulating cytokine release and is also involved in extracellular matrix re-modelling. Elevated levels of granzyme B are also implicated in a number of autoimmune diseases, several skin diseases, and type 1 diabetes. Preferably, human GZMB has an amino acid sequence as deposited in the Uniprot database under accession number P10144, mouse GZMB has an amino acid sequence as deposited in the Uniprot database under accession number P04187. The term also encompasses variants of the aforementioned GZMB proteins.

The term “TIGIT” as used herein refers to “T cell immunoreceptor with Ig and ITIM domains” and is an immune receptor present on T cells and natural killer cells (NK). TIGIT can bind to CD155 (PVR) on dendritic cells (DCs), macrophages, etc. with high affinity, and also to CD112 (PVRL2) with lower affinity. TIGIT can inhibit NK cytotoxicity. Preferably, human TIGIT has an amino acid sequence as deposited in the Uniprot database under accession number Q495A1, mouse TIGIT has an amino acid sequence as deposited in the Uniprot database under accession number P86176. The term also encompasses variants of the aforementioned TIGIT proteins.

The term “KLF2” as used herein refers to “Krüppel-like Factor 2” a member of the Krüppel-like factor family of zinc finger transcription factors. It has been implicated in a variety of biochemical processes in the human body, including lung development, embryonic erythropoiesis, epithelial integrity, T-cell viability, and adipogenesis. Preferably, human KLF2 has an amino acid sequence as deposited in the Uniprot database under accession number Q9Y5W3, mouse KLF2 has an amino acid sequence as deposited in the Uniprot database under accession number Q60843. The term also encompasses variants of the aforementioned KLF2 proteins.

The term “IL-7R” as used herein refers to the “interleukin-7 receptor” which is a cytokine receptor binding to interleukin 7. It is a heterodimeric receptor made up of two different smaller protein chains, i.e. interleukin-7 receptor-α (CD127) and common-γ chain receptor (CD132). The common-γ chain receptors is shared with various cytokines, including interleukin-2, -4, -9, and -15. The Interleukin-7 receptor is expressed on various cell types, including naive and memory T cells and many others. Preferably, human IL-7R has an amino acid sequence as deposited in the Uniprot database under accession number P16871, mouse IL-7R has an amino acid sequence as deposited in the Uniprot database under accession number P16872. The term also encompasses variants of the aforementioned IL-7R proteins.

The term “TCF7” as used herein refers to “Transcription factor 7” a transcription factor protein being involved in T cell development and differentiation, embryonic development, or tumorogenesis. Multiple TCF7 isoforms can be characterized by the full-length isoform (FL-TCF7) as a transcription activator, or dominant negative isoform (dn-TCF7) as a transcription repressor. TCF7 interacts with multiple proteins or target genes and participates in several signal pathways. Preferably, human TCF7 has an amino acid sequence as deposited in the Uniprot database under accession number P36402, mouse TCF7 has an amino acid sequence as deposited in the Uniprot database under accession number Q00417. The term also encompasses variants of the aforementioned TCF7 proteins.

The term “Foxo1” as used herein refers to “Forkhead box protein 01” a transcription factor of the forkhead family which share a characteristic forkhead domain as a common structural element. It is involved in regulation of gluconeogenesis and glycogenolysis by insulin signaling, and is also central to the decision for a pre-adipocyte to commit to adipogenesis. Its activity is dependent on its phosphorylation state. Preferably, human Foxo1 has an amino acid sequence as deposited in the Uniprot database under accession number Q12778, mouse Foxo1 has an amino acid sequence as deposited in the Uniprot database under accession number Q9R1E0. The term also encompasses variants of the aforementioned Foxo1 proteins.

The term “SELL” as used herein refers to “L-selectin”, also known as “CD62L”. It is a cell adhesion molecule found on leukocytes and the preimplantation embryo. It belongs to the selectin family of proteins which recognize sialylated carbohydrate groups. It is cleaved by ADAM17. CD62L is a cell surface component that is a homing receptors that plays important roles in lymphocyte-endothelial cell interactions. The molecule is composed of multiple domains: one homologous to lectins, one to epidermal growth factor, and two to the consensus repeat units found in C3/C4-binding proteins. Preferably, human SELL has an amino acid sequence as deposited in the Uniprot database under accession number P14151, mouse SELL has an amino acid sequence as deposited in the Uniprot database under accession number P18337. The term also encompasses variants of the aforementioned SELL proteins.

More preferably, the hepatic, auto-aggressive CD8 positive (+) PD-1 positive (+) T cells exhibiting traits of activation and exhaustion according to the present invention may exhibit increased expression of at least one further biomarker selected from the group consisting of: Granzyme A, Granzyme K, Prf1, CCL3, CCL4 CCL5, Pdcd1, Mki-67low, IFNγ, Eomes, CD44, and CD244low. Moreover, more preferably, the hepatic, auto-aggressive CD8 positive (+) PD-1 positive (+) T cells exhibiting traits of activation and exhaustion may exhibit decreased expression of at least one further biomarker selected from the group consisting of: CD127, Tbet, and CD62L. The aforementioned biomarkers are well known in the art. Amino acid sequences defining their primary structure can be found in the Uniprot database. Also encompassed in accordance with the present invention are variants of the specific biomarkers as defined elsewhere herein.

Also preferably, the presence, absence or abundance of CD4+ PD-1+ T-cells may be further determined in the method of the present invention. The assessment made can be further strengthened based on the said presence, absence or abundance of CD4+ PD-1+ T-cells in the liver or peripheral blood.

Advantageously, it has been found in the studies underlying the present invention that there is progressive accumulation of exhausted, unconventionally activated CD8+ PD-1+ T-cells in NASH-affected livers or the peripheral blood, i.e. the hepatic auto-aggressive CD8 positive (+) PD-1 positive (+) T cells exhibiting traits of activation and exhaustion. In preclinical models of NASH-induced HCC, therapeutic PD-1-targeted immunotherapy expanded said hepatic auto-aggressive CD8 positive (+) PD-1 positive (+) T cells exhibiting traits of activation and exhaustion within tumors but did not lead to tumor-regression which is indicative of impaired tumor immune-surveillance. When given prophylactically, anti-PD-1 treatment led to elevated NASH-HCC incidence/tumor nodules correlating with numbers of the aforementioned hepatic auto-aggressive CD8 positive (+) PD-1 positive (+) T cells exhibiting traits of activation and exhaustion. Anti-PD1-triggered HCC-increase was prevented by CD8+ T-cell depletion or TNF-neutralization, suggesting a role of hepatic auto-aggressive CD8 positive (+) PD-1 positive (+) T cells exhibiting traits of activation and exhaustion in inducing NASH-HCC, rather than invigorating immune-surveillance. Similar phenotypic and functional profiles of hepatic auto-aggressive CD8+ PD-1+ T-cells were found in human NASH. Characterizing the clinical relevance of these results, a meta-analysis of three randomized phase III trials testing PD-L1/PD-1 inhibitors in more than 1600 patients with advanced HCC revealed that immune therapy did not improve survival in non-viral HCC. In two additional cohorts, patients with NASH-driven HCC and anti-PD-(L)1 treatment displayed significantly reduced overall survival (OS) compared to patients with other etiologies. Collectively, preclinical and clinical data identified non-viral-related HCC, particularly NASH-HCC, as potentially less-responsive to immunotherapy, likely due to NASH-related aberrant T-cell activation causing tissue damage and impaired immune-surveillance. Moreover, hepatic auto-aggressive CD8 positive (+) PD-1 positive (+) T cells exhibiting traits of activation and exhaustion are an apparent subpopulation of CD+ PD1+ T cells found in NASH subjects at various stages. The presence, absence or abundance of these cells can serve as a biomarker for identifying a subject that may benefit from immunotherapy. In particular, CXCR6 and TOX turned out to be particular suitable biomarkers.

Liver cancer develops primarily on the basis of chronic inflammation. The latter can be activated by immunotherapy to induce tumor-regression in a subset of liver cancer patients. However, the identity of responders to immunotherapy for HCC remains elusive. The data underlying this invention identify a non-viral etiology of liver damage and cancer, i.e. NASH, as a predictor of unfavorable outcome in patients treated with immune-checkpoint inhibitors. Better response to immunotherapy in viral-induced HCC patients compared to non-viral HCC patients might be due to the amount or quality of viral-antigens or a different liver micro-environment, possibly not impairing immune-surveillance. The present results also have implications for obese patients with NALFD/NASH suffering from cancer at other organ sites (e.g. melanoma, colon carcinoma, breast cancer) and at risk for liver damage and development of liver cancer in response to systemically applied immunotherapy. Thanks to the present invention a rationale for HCC-patient stratification can be provided according to underlying etiologies in studies testing immunotherapy as primary or adjuvant treatment and a rational basis for HCC patient-stratification according to etiology of liver damage and cancer for future trial designs in personalized cancer therapy was provided in general.

All explanations and definitions of the terms made above apply mutatis mutandis for the following embodiments.

In a preferred embodiment of the method of the present invention, the subject suffers or is suspected to suffer from non-viral-related liver cancer, preferably, hepatocellular carcinoma. In such a case, said treatment response, preferably, is the absence of or an adverse treatment response. The presence of said hepatic auto-aggressive CD8+ PD-1+ T-cells exhibiting traits of activation and exhaustion or CD8+ T cell precursors thereof is, preferably, indicative for an absence of or an adverse treatment response associated with immunotherapy. The adverse treatment response in such a case, preferably, comprises the progression or persistence of liver cancer, preferably, HCC or CCA or intrahepatic metastasis of any origin.

In another preferred embodiment of the method of the present invention, said subject suffers or is suspected to suffer from viral-related liver cancer, preferably, HCC or CCA. Preferably, said treatment response is a therapeutically effective treatment response. The absence of said hepatic auto-aggressive CD8+ PD-1+ T-cells exhibiting traits of activation and exhaustion or CD8+ T cell precursors thereof is, preferably, indicative for a therapeutically effective treatment response associated with immunotherapy. Said therapeutically effective treatment response comprises, preferably, amelioration or cure of liver cancer, preferably, HCC or CCA.

In a further preferred embodiment of the method of the present invention, said subject suffers or is suspected to suffer from non-alcoholic fatty liver disease (NAFLD) or systemic obesity (metabolic syndrome). Preferably, said subject suffers from non-liver cancer susceptible to systemic immunotherapy, preferably, melanoma, prostate cancer, colon cancer, cervix cancer or breast cancer. Moreover, said treatment response is, preferably, an adverse hepatic side effect in said case. The presence of said hepatic auto-aggressive CD8+ PD-1+ T-cells exhibiting traits of activation and exhaustion or CD8+ T cell precursors thereof is, preferably, indicative for an adverse hepatic side effect associated with immunotherapy. Said adverse hepatic side effect, typically, comprises development of liver damage, liver dysfunction or liver cancer, preferably, HCC or CCA.

The present invention also contemplates a method for assessing a treatment response associated with immunotherapy in a subject in need thereof comprising the steps of:
(a) determining data indicating the presence, absence or abundance of (i) hepatic auto-aggressive CD8 positive (+) PD-1 positive (+) T cells exhibiting traits of activation and exhaustion or (ii) CD8+ T cell precursors thereof in a data set comprising imaging data of a subject in need of immunotherapy; and
(b) assessing the treatment response associated with immunotherapy based on the presence, absence or abundance of (i) said hepatic auto-aggressive CD8+ PD-1+ T-cells exhibiting traits of activation and exhaustion or (ii) said CD8+ T cell precursors thereof.

The term “data set comprising imaging data” according to the present invention refers to a collection of imaging data which have been obtained from an in vivo or ex vivo investigation of liver tissue of the subject. The method itself is an ex vivo method which is applied for evaluating said data in order to assess a treatment response associated with immunotherapy in said subject. By determining data in the data set of imaging data which are indicative for the presence, absence or abundance of hepatic auto-aggressive CD8 positive (+) PD-1 positive (+) T cells exhibiting traits of activation and exhaustion or CD8+ T cell precursors thereof an assessment can be made for a treatment response to immunotherapy as discussed elsewhere herein. The imaging data can be obtained by various techniques well known in the art including radiography, magnetic resonance imaging, scintigraphy, SPECT, PET, magnetic particle imaging, Functional near-infrared spectroscopy and the like. The kind of data that are indicative for the presence, absence or abundance of the hepatic auto-aggressive CD8 positive (+) PD-1 positive (+) T cells exhibiting traits of activation and exhaustion or CD8+ T cell precursors thereof depend on the detection technique and the detection agent used. The skilled artisan is well aware of how data indicative for the presence, absence or abundance of hepatic auto-aggressive CD8 positive (+) PD-1 positive (+) T cells exhibiting traits of activation and exhaustion or CD8+ T cell precursors thereof can be identified. Preferably, it is envisaged that data that correspond to the presence, absence or abundance of one or more biomarkers referred to herein are determined. Said data may be data relating to a detectable signal elicited by a detectable label as specified herein.

The present invention also relates to a method for recommending immunotherapy for a subject comprising assessing the treatment response to immunotherapy for said subject by carrying out the aforementioned method of the invention and, recommending immunotherapy for said subject if the subject is assessed to have no non-treatment response, no adverse treatment response, a therapeutically effective treatment response and/or no adverse hepatic side effect.

Moreover, the present invention also relates to a method for treating a subject by immunotherapy comprising assessing the treatment response to immunotherapy for said subject by carrying out the aforementioned method of the invention and, administering immunotherapy to said subject if the subject is assessed to have no non-treatment response, no adverse treatment response, a therapeutically effective treatment response and/or no adverse hepatic side effect.

The present invention also relates to a device for assessing a treatment response associated with immunotherapy in a subject in need thereof comprising:
(a) an analyzing unit capable of determining (i) hepatic auto-aggressive CD8 positive (+) PD-1 positive (+) T cells exhibiting traits of activation and exhaustion or (ii) CD8+ T cell precursors thereof in a sample of a subject in need of immunotherapy; and
(b) an evaluation unit comprising a data processor capable of assessing the treatment response associated with immunotherapy based on the presence, absence or abundance of (i) said hepatic auto-aggressive CD8+ PD-1+ T-cells exhibiting traits of activation and exhaustion or (ii) said CD8+ T cell precursors thereof.

The term “device” as used herein refers to a system comprising the aforementioned units operatively linked to each other as to allow the determination of the presence, absence or abundance of biomarkers and evaluation thereof according to the method of the invention such that an assessment can be provided.

The analyzing unit, typically, comprises at least one reaction zone having a biomarker detection agent for the first and second biomarker and, preferably also the third biomarker, in immobilized form on a solid support or carrier which is to be contacted to the sample. Moreover, in the reaction zone, it is possible to apply conditions which allow for the specific binding of the detection agent(s) to the biomarkers comprised in the sample. The reaction zone may either allow directly for sample application or it may be connected to a loading zone where the sample is applied. In the latter case, the sample can be actively or passively transported via the connection between the loading zone and the reaction zone to the reaction zone. Moreover, the reaction zone shall be also connected to a detector. The connection shall be such that the detector can detect the binding of the biomarkers to their detection agents. Suitable connections depend on the techniques used for measuring the presence or amount of the biomarkers. For example, for optical detection, transmission of light may be required between the detector and the reaction zone while for electrochemical determination a fluidal connection may be required, e.g., between the reaction zone and an electrode. The detector shall be adapted to detect determine the amount of the biomarkers. The determined amount can be subsequently transmitted to the evaluation unit.

Said evaluation unit comprises a data processing element, such as a computer, with an implemented algorithm for determining the amount present in the sample. The processing unit as referred to in accordance with the method of the present invention, typically, comprises a Central Processing Unit (CPU) and/or one or more Graphics Processing Units (GPUs) and/or one or more Application Specific Integrated Circuits (ASICs) and/or one or more Tensor Processing Units (TPUs) and/or one or more field-programmable gate arrays (FPGAs) or the like. A data processing element may be a general purpose computer or a portable computing device, for example. It should also be understood that multiple computing devices may be used together, such as over a network or other methods of transferring data, for performing one or more steps of the methods disclosed herein. Exemplary computing devices include desktop computers, laptop computers, personal data assistants (“PDA”), cellular devices, smart or mobile devices, tablet computers, servers, and the like. In general, a data processing element comprises a processor capable of executing a plurality of instructions (such as a program of software). The evaluation unit, typically comprises or has access to a memory. A memory is a computer readable medium and may comprise a single storage device or multiple storage devices, located either locally with the computing device or accessible to the computing device across a network, for example. Computer-readable media may be any available media that can be accessed by the computing device and includes both volatile and non-volatile media. Further, computer readable-media may be one or both of removable and non-removable media. By way of example, and not limitation, computer-readable media may comprise computer storage media. Exemplary computer storage media includes, but is not limited to, RAM, ROM, EEPROM, flash memory or any other memory technology, CD-ROM, Digital Versatile Disk (DVD) or other optical disk storage, magnetic cassettes, magnetic tape, magnetic disk storage or other magnetic storage devices, or any other medium which can be used for storing a plurality of instructions capable of being accessed by the computing device and executed by the processor of the computing device. The evaluation unit may also comprise or has access to an output device. Exemplary output devices include fax machines, displays, printers, and files, for example. According to some embodiments of the present disclosure, a computing device may perform one or more steps of a method disclosed herein, and thereafter provide an output, via an output device, relating to a result, indication, ratio or other factor of the method.

Preferably, said device is adopted to carry out the method of the present invention.

The present invention, furthermore, relates to a device for assessing a treatment response associated with immunotherapy in a subject in need thereof comprising:
(a) an analyzing unit capable of determining data indicating the presence, absence or abundance of (i) hepatic auto-aggressive CD8 positive (+) PD-1 positive (+) T cells exhibiting traits of activation and exhaustion or (ii) CD8+ T cell precursors thereof in a data set comprising imaging data of a subject in need of immunotherapy; and
(b) an evaluation unit capable of assessing the treatment response associated with immunotherapy based on the presence, absence or abundance of (i) said hepatic auto-aggressive CD8+ PD-1+ T-cells exhibiting traits of activation and exhaustion or (ii) said CD8+ T cell precursors thereof.

Preferably, said device is adopted to carry out the method of the invention.

Yet, the present invention provides a kit for assessing a treatment response associated with immunotherapy in a subject in need thereof comprising at least one detection agent that allows for specific determination of (i) hepatic auto-aggressive CD8 positive (+) PD-1 positive (+) T cells exhibiting traits of activation and exhaustion or (ii) CD8+ T cell precursors.

The term “kit” as used herein refers to collection of the aforementioned components, typically, provided in separately or within a single container. The container also typically comprises instructions for carrying out the method of the present invention. These instructions may be in the form of a manual or may be provided by a computer program code which is capable of carrying out or supports the determination of the biomarkers referred to in the methods of the present invention when implemented on a computer or a data processing device. The computer program code may be provided on a data storage medium or device such as an optical storage medium (e.g., a Compact Disc) or directly on a computer or data processing device or may be provided in a download format such as a link to an accessible server or cloud. Moreover, the kit may, usually, comprise standards for reference amounts of biomarkers for calibration purposes as described elsewhere herein in detail. The kit according to the present invention may also comprise further components which are necessary for carrying out the method of the invention such as solvents, buffers, washing solutions and/or reagents required for detection of the released second molecule. Further, it may comprise the device of the invention either in parts or in its entirety.

Preferably, said at least one detection agent that allows for specific detection of a biomarker selected from the group consisting of: TOX, CXCR6, TNFα, LAG3, GZMB (Granzyme B) TIGIT, KLF2, IL-7R, TCF7, Foxo1 and SELL. More preferably, said detection agent is an antibody, aptamer or nucleic acid molecule that specifically binds to the biomarker or nucleic acid transcript encoding it.

The following embodiments are particularly envisaged embodiments in accordance with the present invention.

Embodiment 1. A method for assessing a treatment response associated with immunotherapy in a subject in need thereof comprising the steps of:
(a) determining (i) hepatic auto-aggressive CD8 positive (+) PD-1 positive (+) T cells exhibiting traits of activation and exhaustion or (ii) CD8+ T cell precursors thereof in a sample of a subject in need of immunotherapy; and
(b) assessing the treatment response associated with immunotherapy based on the presence, absence or abundance of (i) said hepatic auto aggressive CD8+ PD-1+ T-cells exhibiting traits of activation and exhaustion or (ii) said CD8+ T cell precursors thereof.

Embodiment 2. The method of embodiment 1, wherein said sample is a liver biopsy sample.

Embodiment 3. A method for assessing a treatment response associated with immunotherapy in a subject in need thereof comprising the steps of:
(a) determining data indicating the presence, absence or abundance of (i) hepatic auto-aggressive CD8 positive (+) PD-1 positive (+) T cells exhibiting traits of activation and exhaustion or (ii) CD8+ T cell precursors thereof in a data set comprising imaging data of a subject in need of immunotherapy; and
(b) assessing the treatment response associated with immunotherapy based on the presence, absence or abundance of (i) said hepatic auto-aggressive CD8+ PD-1+ T-cells exhibiting traits of activation and exhaustion or (ii) said CD8+ T cell precursors thereof.

Embodiment 4. The method of any one of embodiments 1 to 3, wherein said subject suffers or is suspected to suffer from non-viral-related liver cancer, preferably, hepatocellular carcinoma.

Embodiment 5. The method of any one of embodiments 1 to 4, wherein said treatment response is the absence of or an adverse treatment response.

Embodiment 6. The method of embodiment 5, wherein the presence of (i) said hepatic auto-aggressive CD8+ PD-1+ T-cells exhibiting traits of activation and exhaustion or (ii) said CD8+ T cell precursors thereof is indicative for an absence of or an adverse treatment response associated with immunotherapy.

Embodiment 7. The method of embodiments 5 or 6, wherein said adverse treatment response comprises the progression or persistence of liver cancer, preferably, hepatocellular carcinoma (HCC) or cholangiocarcinoma (CCA) or intrahepatic metastasis of any origin.

Embodiment 8. The method of any one of embodiments 1 to 3, wherein said subject suffers or is suspected to suffer from viral-related liver cancer, preferably, HCC or CCA.

Embodiment 9. The method of embodiment 8, wherein said treatment response is a therapeutically effective treatment response.

Embodiment 10. The method of embodiment 9, wherein the absence of (i) said hepatic auto-aggressive CD8+ PD-1+ T-cells exhibiting traits of activation and exhaustion or (ii) said CD8+ T cell precursors thereof is indicative for a therapeutically effective treatment response associated with immunotherapy.

Embodiment 11. The method of embodiments 9 or 10, wherein said therapeutically effective treatment response comprises amelioration or cure of liver cancer, preferably, HCC or CCA.

Embodiment 12. The method of any one of embodiments 1 to 3, wherein said subject suffers or is suspected to suffer from non-alcoholic fatty liver disease (NAFLD) or systemic obesity (metabolic syndrome).

Embodiment 13. The method of embodiment 12, wherein said subject suffers from non-liver cancer susceptible to systemic immunotherapy, preferably, melanoma, prostate cancer, colon cancer, cervix cancer or breast cancer.

Embodiment 14. The method of embodiments 12 or 13, wherein said treatment response is an adverse hepatic side effect.

Embodiment 15. The method of embodiment 14, wherein the presence of (i) said hepatic auto-aggressive CD8+ PD-1+ T-cells exhibiting traits of activation and exhaustion or (ii) said CD8+ T cell precursors thereof is indicative for an adverse hepatic side effect associated with immunotherapy.

Embodiment 16. The method of embodiments 14 or 15, wherein said adverse hepatic side effect comprises development of liver damage, liver dysfunction or liver cancer, preferably, HCC or CCA.

Embodiment 17. The method of any one of embodiments 1 to 16, wherein said immunotherapy involves PD-1 and/or PD-L1 targeted immunotherapy.

Embodiment 18. The method of any one of embodiments 1 to 17, wherein said subject is a mammal, preferably, a human.

Embodiment 19. The method of any one of embodiments 1 to 18, wherein said hepatic auto-aggressive CD8+ PD-1+ T cells exhibiting traits of activation and exhaustion exhibit an increased expression compared to control CD8+ T cells of at least one biomarkers selected from the group consisting of: TOX, CXCR6, TNFα, LAG3, GZMB (Granzyme B) and TIGIT, more preferably, CXCR6 and TOX.

Embodiment 20. The method of any one of embodiments 1 to 19, wherein said hepatic auto-aggressive CD8+ PD-1+ T cells exhibiting traits of activation and exhaustion exhibit a reduced expression compared to control CD8+ T cells of at least one biomarker selected from the group consisting of: KLF2, IL-7R, TCF7, Foxo1 and SELL.

Embodiment 21. The method of any one of embodiments 1 to 18, wherein said CD8+ T cell precursors are characterized by at least one biomarker selected from the group consisting of: TCF7, SELL, and IL-7R.

Embodiment 22. The method of embodiment 21, wherein said CD8+ T cell precursors exhibit a change in expression over time of at least one biomarker selected from the group consisting of: TOX, CXCR6, TNFα, LAG3, GZMB (Granzyme B) TIGIT, KLF2, IL-7R, TCF7, Foxo1 and SELL, more preferably, CXCR6 and TOX.

Embodiment 23. The method of embodiment 22, wherein (i) said change is a decrease in expression over time if said at least one biomarker is selected from the group consisting of KLF2, IL-7R, TCF7, Foxo1 and SELL; and (ii) said change is an increase in expression over time if said at least one biomarker is selected from the group consisting of TOX, CXCR6, TNFα, LAG3, GZMB (Granzyme B) and TIGIT, more preferably, CXCR6 and TOX.

Embodiment 24. A method for recommending immunotherapy for a subject comprising assessing the treatment response to immunotherapy for said subject by carrying out the method of any one of embodiments 1 to 23 and, recommending immunotherapy for said subject if the subject is assessed to have no non-treatment response, no adverse treatment response, a therapeutically effective treatment response and/or no adverse hepatic side effect.

Embodiment 25. A method for treating a subject by immunotherapy comprising assessing the treatment response to immunotherapy for said subject by carrying out the method of any one of embodiments 1 to 23 and, administering immunotherapy to said subject if the subject is assessed to have no non-treatment response, no adverse treatment response, a therapeutically effective treatment response and/or no adverse hepatic side effect.

Embodiment 26. A device for assessing a treatment response associated with immunotherapy in a subject in need thereof comprising:
(a) an analyzing unit capable of determining (i) hepatic auto-aggressive CD8 positive (+) PD-1 positive (+) T cells exhibiting traits of activation and exhaustion or (ii) CD8+ T cell precursors thereof in a sample of a subject in need of immunotherapy; and
(b) an evaluation unit comprising a data processor capable of assessing the treatment response associated with immunotherapy based on the presence, absence or abundance of (i) said hepatic auto-aggressive CD8+ PD-1+ T-cells exhibiting traits of activation and exhaustion or (ii) said CD8+ T cell precursors thereof.

Embodiment 27. The device of embodiment 26, wherein said device is adopted to carry out the method of any one of embodiments 1, 2 or, as far as dependent on embodiment 1 or 2, embodiments 4 to 25.

Embodiment 28. A device for assessing a treatment response associated with immunotherapy in a subject in need thereof comprising:
(a) an analyzing unit capable of determining data indicating the presence, absence or abundance of (i) hepatic auto-aggressive CD8 positive (+) PD-1 positive (+) T cells exhibiting traits of activation and exhaustion or (ii) CD8+ T cell precursors thereof in a data set comprising imaging data of a subject in need of immunotherapy; and
(b) an evaluation unit capable of assessing the treatment response associated with immunotherapy based on the presence, absence or abundance of (i) said hepatic auto-aggressive CD8+ PD-1+ T-cells exhibiting traits of activation and exhaustion or (ii) said CD8+ T cell precursors thereof.

Embodiment 29. The device of embodiment 28, wherein said device is adopted to carry out the method of embodiment 3 or, as far as dependent on embodiment 3, any one of embodiments 4 to 25.

Embodiment 30. A kit for assessing a treatment response associated with immunotherapy in a subject in need thereof comprising at least one detection agent that allows for specific determination of (i) hepatic auto-aggressive CD8 positive (+) PD-1 positive (+) T cells exhibiting traits of activation and exhaustion or (ii) CD8+ T cell precursors.

Embodiment 31. The kit of embodiment 30, wherein said at least one detection agent that allows for specific detection of a biomarker selected from the group consisting of: TOX, CXCR6, TNFα, LAG3, GZMB (Granzyme B) TIGIT, KLF2, IL-7R, TCF7, Foxo1 and SELL, more preferably, CXCR6 and TOX.

Embodiment 32. The kit of embodiment 31, wherein said detection agent is an antibody, aptamer or nucleic acid molecule that specifically binds to the biomarker or nucleic acid transcript encoding it.

All references cited throughout this specification and herein below are herewith incorporated by reference with respect to the specifically mentioned disclosure content as well as in their entireties.

### FIGURES

FIG. 1: (a) Histological analysis of liver steatosis upon different NASH diets or a control diet. ND: normal diet; CD-HFD: choline deficient high fat diet. WD: Western diet; (b) Liver damage characterization (upper panel; ALT: Amino-transferase) and (c) NAFLD activity score analysis (lower panel) of mice on different NASH diets or a control diet; (d) Flow cytometry analysis of all CD45+ immune cells. Significant Increase of the T-cell population (CD8+ PD1+, arrow) is seen; (e) CD8 and PD-1 staining (right image) and quantification by liver 5 immunohistochemistry (n=5-6 mice/group); (f) Immunofluorescence-based detection of PD-1, CD8 and CD4 cells (n=3 mice/group); (g) Gene set enrichment analysis of hepatic CD8+ PD-1+6 T-cells sorted TCRβ+ cells by mass spectrometry (n=4-6 mice/group; (h) tSNE of TCRβ+ cells that were analysed by single cell RNA Seq.; (i) Differential gene expression of TCRfβ+ cells that were analysed by single cell RNA Seq. (j) RNA velocity indicating transcriptional activity, gene expression, and the trajectory of CD8+ cells by scRNA-seq from 12 months ND or CD-HFD-fed mice (n=3 mice/group); (k) Scheme for anti-PD-1 application and tumor incidence after 8 weeks of treatment of 15 months CD-HFD (tumor/lesion size and tumor load: n=7-9 mice/group; tumor incidence: CD-HFD n=17 tumors/lesions in 22 mice; CD-HFD+α-PD-1 n=10 tumors/lesions in 10 mice); (1) Magnetic resonance imaging of livers of mice after 13-months CD-HFD-fed mice followed by 8 weeks of anti-PD-1 treatment (n=4 mice). Lines: tumor nodule. Scale bar: 10 mm; (m) Livers macroscopy after diet with or without immunotherapy. Arrowheads: tumor/lesions. Scale bar: 10 mm; (n) Liver CD8+ cell quantification by immunohistochemistry. (n=3-13 mice/group; intra-tumoral staining: n=8-11 mice/group); (o) Quantification of liver tumor tissue for CXCR6 expression by mRNA mRNA in situ hybridization. Scale bars: 100 μm. Arrowheads: positive cells.

FIG. 2: Resident-like CD8+ PD-1+ T-cells drive hepatocarcinogenesis in a TNF-dependent manner upon anti-PD-1 treatment in NASH (a) RNA-velocity analyses of scRNA-seq data showing expression and (b) correlation of expression along the latent-time of selected genes. (c) PCA plot of hepatic or peripheral blood derived CD8+ or CD8+ PD-1+ T-cells sorted TCRβ+ cells by mass spectrometry of 12 months ND, CD-HFD or CD-HFD fed mice+8 weeks treatment by α-PD-1 antibodies. (d) UMAP representation showing the FlowSOM-guided clustering, heatmap showing the median marker expression, and (e) quantification of hepatic or peripheral blood derived CD8+ T-cells of 12 months ND, CD-HFD+IgG or CD-HFD-fed mice +8 weeks treatment by α-PD-1 antibodies. (f) Quantification of CellCNN analyzed flow cytometry data of hepatic or peripheral blood derived CD8+ T-cells of 12 months CD-HFD+IgG or CD-HFD-fed mice+8 weeks treatment by α-PD-1 antibodies. (g) ALT, (h) NAS evaluation (i) Quantification of hepatic or peripheral blood derived CD8+ PD-1+CXCR6+ and CD8+ PD-1+ TNF+ T-cells of 12 months ND, CD-HFD, CDHFD-fed mice+8 weeks treatment by α-PD-1, α-PD-1/α-CD8, α-TNF, α-PD-1/α-TNF, α-CD4, or α-PD-1/α-CD4 antibodies. (j) Quantification of tumor incidence.

FIG. 3: PD-1 and PD-L1 targeted immunotherapy in advanced HCC has a distinct effect depending on disease etiology(a) Meta-analysis of 1656 patients. Immunotherapy was initially assessed and then analyzed according to disease etiology: non-viral (NASH and alcohol intake) vs viral. Separate meta-analyses were subsequently performed for each of the three etiologies: non-viral (NASH and alcohol intake), HCV and HBV. (b) NAFLD is associated with a worse outcome in patients with hepatocellular carcinoma (HCC) treated with PD-(L)1-targeted immunotherapy. A total of 130 patients with advanced HCC received PD-(L)1-targeted immunotherapy. (c) Validation cohort of patients with HCC treated with PD-(L)1-targeted immunotherapy. A total of 118 patients with advanced HCC received PD-(L)1-targeted immunotherapy.

FIG. 4: Investigating the CD8+ population reveals that only a subpopulation of CD8+ PD1+ T cells exhibits traits of exhaustion and activation. (a) UMAP representation showing the FlowSOM-guided clustering, heatmap showing the median marker expression, (b) UMAP representation of individual markers of exhaustion and activation within the CD8+ PD1+ T cell population reveals different subpopulations.

### EXAMPLES

The Examples shall merely illustrate the invention and shall, by no means, construed as limiting its scope.

### Example 1: Methods and Materials

Mice, Diets, and Treatments

Standard mouse diet feeding (ad libitum water and food access) and treatment regimens were described previously. Male mice were housed (constant temperature of 20-24° C. and 45-65% humidity with a 12 h light cycle) at the German Cancer Research Center (DKFZ). Animals were maintained under specific pathogen-free conditions and experiments were performed in accordance to German Law (G11/16, G129/16, G7/17). Tissues from inducible knock-in mice expressing the human unconventional prefoldin RPB5 interactor were received. The plasmids for hydrodynamic tail-vein-delivery have been previously described. For interventional studies, male CD-HFD-fed mice were treated with bi-weekly for 8 weeks intra-venous injections of 25 μg CD8-depleting antibody (Bioxcell, 2.43), 50 μg NK1.1-depleting antibody (Bioxcell, PK136), 300 μg anti-PD-L1 (Bioxcell, 10F.9G2), 200 μg anti-TNF (Bioxcell, XT3.11), 100 μg anti-CD4 (Bioxcell, GK1.5), or 150 μg anti-PD-1 (Bioxcell, RMP1-14). PD-1−/− mice were thankfully provided by G. Tiegs and K. Neumann. Mice (Extended Data 3g) anti-PD-1 antibody (Bioxcell, RMP1-14) or Isotype Control (Bioxcell, 2A3) at an initial dose of 500 μg i.p. followed by doses of 200 μg bi-weekly for 8 weeks. Mice (Extended Data 3h) were treated i.p. with anti-PD-1 (200 μm, Bioxcell, RMP1-14) or IgG (200 μg, Bioxcell, LTF-2). Treatment regimen for Extended Data 3i was described in the prior art. Intraperitoneal glucose tolerance test and measurement of serum parameters were described previously.

Magnetic Resonance Imaging

MRI was done in the small animal imaging core facility in DKFZ using a Bruker BioSpec 9.4 Tesla (Ettlingen, Germany). Mice were anesthetized with 3.5% sevoflurane, and imaged with T2 weighted imaging using a T2_TurboRARE sequence: TE=22 ms, TR=2200 ms, field of view (FOV) 35×35 mm, slice thickness 1 mm, averages=6Scan Time 3 m18 s, echo spacing 11 ms, rare factor 8, slices 20, image size 192×192, resolution 0.182×0.182 mm.

Multiplex ELISA

Liver homogenates were prepared analogously to western-blot and cytokines/chemokines were analyzed on a customized ELISA according to the manufacturer's manual (Meso Scale Discovery, U-PLEX Biomarker group 1, K15069L-1).

Flow Cytometry and FACS: Isolation and Staining of Lymphocytes

After perfusion, and mechanical dissection, livers were incubated for up to 35 min at 37° C. with Collagen IV (60U f.c.) and DNase I (25 μm/ml f.c.)), 100 μm filtered, washed with RPMI1640 (#11875093). Next, 2-step Percoll gradient (25%/50% Percoll/HBSS), centrifugation for 15 min/1800 g/4° C. enriched leukocytes were collected, washed, and counted. For restimulation, cells were incubated for 2 h, 37° C., 5% CO2 using 1:500 Biolegend's Cell Activation Cocktail (with Brefeldin A) (#423304) and 1:1000 Monensin Solution (#420701). Live/Dead discrimination by using DAPI or ZombieDyeNIR according to the manufacturer's instructions with subsequent staining of titrated antibodies. Samples for flow cytometric activated cell sorting (FACS) were sorted, samples for flow cytometry were fixed using eBioscience IC fixation (#00-8222-49) or Foxp3 Fix/Perm kit (#00-5523-00) according to the manufacturer's instruction. Intracellular staining was performed in eBioscience Perm buffer (#00-8333-56). Cells were analyzed using BD FACSFortessa or BD FACSSymphony and data were analyzed using FlowJo (v10.6.2). For sorting, FACS Aria II and FACSAria FUSION in collaboration with the DKFZ FACS core facility were used. For UMAP/FlowSOM plots, BD FACSymphony data (mouse and human) were exported from FlowJo (v10). Analyses was performed as described elsewhere in the prior art.

Single-Cell RNA Sequencing and Metacell Analysis (Mouse)

Single-cell capturing for scRNA-seq and library preparation was described previously. Libraries (pooled at equimolar concentration) were sequenced on an Illumina NextSeq 500 at a median sequencing depth of ˜40,000 reads/cell. Sequences were mapped to the mouse (mm10), using HISAT (version 0.1.6); reads with multiple mapping positions were excluded. Reads were associated with genes if they were mapped to an exon, using the Ensembl gene annotation database (Emb1 release 90). Exons of different genes that shared a genomic position on the same strand were considered as a single gene with a concatenated gene symbol. The level of spurious UMIs in the data was estimated by using statistics on empty MARS-seq wells and excluded rare cases with estimated noise >5% (median estimated noise overall experiments was 2%). Removal of specific mitochondrial genes, immunoglobulin genes, genes linked with poorly supported transcriptional models (annotated with the prefix “Rp-”), and cells with less than 400 UMIs. Gene features were selected using Tvm=0.3 and a minimum total UMI count >50. Hierarchical clustering of the correlation matrix between those genes (filtering genes with low coverage and computing correlation using a down-sampled UMI matrix) and selected the gene clusters that contained anchor genes. K=50, 750 bootstrap iterations, and otherwise standard parameters were used. Subsets of T-cells were obtained by hierarchical clustering of the confusion matrix and supervised analysis of enriched genes in homogeneous groups of metacells.

Velocity and Correlation Analyses of scRNA-Seq Data

Velocyto (0.6) was used to estimate the spliced/unspliced counts from the pre-aligned bam files. RNA velocity, latent time, root, and terminal states were calculated using the dynamical velocity model from scvelo (0.2.2). Kendall's rank correlation coefficient was used to correlate the expression patterns of biologically significant genes with latent time.

Preparation for Mass Spectrometry, Data Acquisition, and Data Analysis

After FACS purification, cells were resuspended in 50% (vol/vol) 2,2,2-Trifluoroethanol in PBS pH 7.4 buffer and lysed by repeated sonication, and freeze-thaw cycles. Proteins were denatured at 60° C. for 2 h, reduced using dithiothreitol at a final concentration of 5 mM (30 min at 60° C.), cooled to RT, alkylated using iodoacetamide at 25 mM (30 min at RT in the dark), and diluted 1:5 using 100 mM of ammonium bicarbonate, pH 8.0. Proteins were digested overnight by trypsin (1:100 ratio, 37° C.), desalted using C18 based stage-tips, dried under vacuum, resuspended in 20 μL of HPLC-grade water with 0.1% formic acid, and measured using A380. 0.5 ug of peptides—separated on a 50-cm—were used for proteomic analysis, which was a C18 column using a nano liquid chromatography system (EASY-nLC 1200, Thermo Fisher Scientific). Peptides were eluted using a gradient of 5-30% buffer B (80% acetonitrile and 0.1% formic acid) at a flow rate of 300 nL/min at a column temperature of 55° C. Data were acquired by data-dependent Top15 acquisition using a high-resolution orbitrap tandem mass spectrometer (QExactive HFX, Thermo Scientific). All MS1 scans were acquired at 60,000 resolution with AGC target of 3e6, and MS2 scans were acquired at 15,000 resolution with AGC target of 1e5 and maximum injection time of 28 ms. Analyses was performed using MaxQuant (1.6.7.0), mouse UniProt Isoform fasta (Version: 2019-02-21, number of sequences 25,233) as a source for protein sequences. 1% FDR was used for controlling at the peptide and protein level, with a minimum of two peptides needed for consideration of analysis. Gene set enrichment analysis was performed using ClusterProfiler (3.18)42 and gene sets obtained from WikiPathway (wikipathways.org) and MSigDB (broadinstitute.org/msigdb).

Histology, Immunohistochemistry, Scanning, and Automated Analysis

Histology, immunohistochemistry, scanning, and automated analysis was described previously. Antibodies used in the experiments are all known in the art. For immunofluorescence staining, IHC established antibodies were used, coupled with the AKOYA Biosciences Opal fluorophore kit (Opal 520 FP1487001KT, Opal 540 FP1494001KT, Opal 620 FP1495001KT). For mRNA 5 in situ hybridization freshly non-baked 5 μm FFPE were cut and stained according to manufacturer's (ACD biotech) protocol for manual assay RNAscope, using probes TNF (311081) and CXCR6 (871991).

Isolation of RNA and Library Preparation for Bulk RNA Sequencing

RNA isolation and library preparation for bulk 3′-sequencing of poly(A)-RNA was described previously. Using the with the Feature Extraction software (11.0.1.1, Agilent Technologies), gencode gene annotations version M18 and the mouse reference genome major release GRCm38 were derived from (https://www.gencodegenes.org/). Dropseq tools v1.1247 were used for mapping the raw sequencing data to the reference genome. Resulting UMI filtered count matrix was imported into R v3.4.4. Prior differential expression analysis with Limma v3.40.648 sample-specific weights were estimated and used as coefficients alongside the experimental groups as a covariate during model fitting with Voom. T-test was used for determining differentially (p-value below 0.05) regulated genes between all possible experimental groups. Gene set enrichment analysis was conducted with the pre-ranked GSEA method44 within the MSigDB Reactome, KEGG, and Hallmark databases (broadinstitute.org/msigdb). Raw sequencing data are available under the accession number PRJEB36747.

Stimulation of CD8 T-Cells

Stimulation of CD8 T-cells is described in the prior art.

Flow Cytometry of Human Biopsies

Analysis of patient material was performed on liver tissue (needle biopsies or resected tissue, BIOFACS Study KEK 2019-00114), which were obtained from the patient collection n AC-2019-3627 (CRB03) from the biological resource center of CHU Grenoble-Alpes (n BRIF BB-0033-00069). Tissue samples were minced using scalpels, incubated (1 mg/mL collagenase IV (Sigma Aldrich), 0.25 μg/mL DNase (Sigma Aldrich), 10% FCS (Thermo Fisher Scientific), RPMI 1640 (Seraglob)) for 30 min at 37° C., stopping enzymatic reaction by 2 mM EDTA (StemCell Technologies, Inc) in PBS. After filtering through a 100 μm cell strainer. NexT-cells were resuspended in FACS buffer (PBS, EDTA 2 mM, FCS 0.5%) with Human TruStain FcX^{TM} (Fc Receptor Blocking Solution) (Biolegend) and incubated for 15 min at 4° C. and stained with antibodies. Flow cytometry of human samples (Extended Data 9d) was approved by the local ethical committee (AC-2014-2094 n 03).

High-Throughput RNA Sequencing of Human Specimen

As previously reported, RNA sequencing analysis was performed using the data from 206 snap-frozen biopsy samples from 206 patients diagnosed with NAFLD in France, Germany, Italy, and the UK and enrolled in the European NAFLD Registry (GSE135251). Samples were scored for NAS by two pathologists. Alternate diagnoses were excluded, including excessive alcohol intake (30 g per day for males, 20 g for females), viral hepatitis, autoimmune liver diseases, and steatogenic medication use. Patient samples were grouped: NAFL (n=51) and NASH with different fibrosis stages ranging from F0/1 (n=34), F2 (n=53), F3 (n=54) to F4 (n=14). Collection and use of data of the European NAFLD Registry were approved by the relevant local and/or national Ethical Review Committee. A correction for sex, batch, and center effect was implemented. Pathway enrichment and visualization were described in the prior art.

Immunohistochemistry of NAFLD/NASH Cohort

65 human FFPE biopsies from patients with NAFLD were included. Sequential slides were immunostained with antibodies against human CD8 (Roche, SP57, ready-to-use), PD-1 (Roche; NAT105, ready-to-use), and CD4 (Abcam, ab133616, 1:500). All staining was performed on the VENTANA BenchMark autostainer at 37° C. Immunopositive cells were quantified at 400× magnification in the portal tract and the adherent parenchyma.

Isolation of Cells for Single-Cell RNA-Seq Data Analysis (Human)

Analyses of liver samples from patients undergoing bariatric surgery at the Department of Surgery at Heidelberg University Hospital (S-629/2013) by formalin-fixed/paraffin-embedded for pathological evaluation and single-cell were generated by mincing, performing Miltenyi tumor dissociation kit (Cat no. 130-095-929) as per manufacturer's instructions, filter through a 70 um cell strainer and washing. ACK lysis buffer (Thermo Fischer Scientific Cat no. A1049201) was performed, and samples were stored in FBS+ 20% DMSO till further processing (single-cell RNAseq analysis and mass cytometry). Cells were thawed in 37° C. water bath, washed with PBS+ 0.05 mM EDTA (10 min, 300 g, +4° C.), FC-block (10 min, +4° C.), stained with CD45-PE (3 μl, H130, #12-0459-42) and Live/Dead discrimination (1:1000, Thermofischer, L34973), washed and sorted on a FACSAria FUSION in collaboration with the DKFZ FACS. Library generation was performed according to the manufacturer's protocol (Chromium Next EM Single Cell 3′ GEM, 10000128), sequencing was performed on an Illumina NovaSeq 6000. De-multiplexing and barcode processing was performed using the Cell Ranger Software Suite (Version 4.0.0) and reads were aligned to human GRCh3854. Gene-barcode matrix containing cell barcodes and gene expression counts was generated by counting the single-cell 3′ UMIs, imported into R (v4.0.2) where quality control and normalization were executed using Seurat v355. Cells with more than 10% mitochondrial genes, fewer than 200 genes per cell, or more than 6000 genes per cell were excluded. Matrices from 10 samples were integrated with Seurat v3 to remove batch effects across samples. PCA analysis of filtered gene-barcode matrices of all CD3+ cells, visualized by UMAP (top 50 principal components) and identification of major cell types using the highly variable features and indicative markers was performed. Besides, pairwise combinations of CD4+ T-cells vs CD4+ PD-1+ T-cells and CD8+ T-cells vs CD8+ PD-1+ T-cells were performed using the results of differential expression analysis by DESeq2 (v1.28.1), setting CD4+/CD8+ T-cells as controls. Volcano plots were then generated using EnhancedVolcano (v1.6.0) to visualize the results of differential expression analysis.

Mass Cytometry Data Analysis (Human)

Antibody conjugates for mass cytometry were purchased from Fluidigm, generated in-house using antibody labeling kits (Fluidigm X8, MCP9), or as described before. Antibody cocktails for mass cytometry were cryopreserved as described before. Isolation of cells is described in the paragraph “Isolation of cells for single-cell RNA-seq data analysis (human)”. Cells were thawed, transferred into RPMI+Benzonase (14 ml RPMI+0.5 μl Benzonase), and centrifuged for 5 min at 500 μg. The cell pellet was resuspended in 1 ml of CSM-B (CSM (PBS 0.5% BSA 0.02% sodium azide)+1 ul of Benzonase), filtered through a 30 μm cell strainer, adjusted to 3 ml, counted, resuspended in 35 μl CSM-B, incubated for 45 min at 4° C. and 100 μl of CSM-B were added. Cells were pooled and stained with a surface antibody cocktail for 30 min, 4° C. Dead cell discrimination was performed with mDOTA-103Rh (5 min, RT). For intracellular staining, Foxp3 intracellular staining kit from Miltenyi Biotec was used as per the manufacturer's instructions, followed by staining for intracellular targets for 30 min, RT. Cells were washed, resuspended in 1 ml of iridium intercalator solution, and incubated for 25 min, RT. Cells were washed with CSM, PBS, MilliQ water, adjusted at a final concentration of 7.5×105 cells/mL and supplemented with 4-element EQ beads. The sample was acquired on a Helios mass cytometer and raw data were EQBead-normalized using Helios mass cytometer and Helios instrument software (version 6.7). Compensation was performed in CATALYST (v1.86)61 and FlowCore (1.50.0). De-barcoding and gating of single, live CD45+ cells were performed using FlowJo (v10.6.2). Then, data of CD45+ cells were imported into Cytosplore 2.3.1 and transformed using the arcsinh(5) function. Major immune cell lineages were identified at the first level of a 2-level hierarchical stochastic neighbor embedding (HSNE) analysis with default perplexity and iteration settings. HSNE with the same parameters was run on CD3+ cells to identify T-cell phenotypes. Gaussian mean shift clustering was performed in Cytosplore and a heatmap of arcsinh(5)—transformed expression values of all antibody targets was generated. Cell type identification was based on the transformed expression values and clusters showing high similarity were merged manually.

Histological and Immunohistochemical Analysis of NASH/HCC Cohort

4 healthy samples, 16 NASH cases, and non-tumoral tissue adjacent to HCC tumors from patients of the following etiologies were selected: NASH (n=26), viral hepatitis (n=19 HCV, n=3 HBV), alcohol (n=5), and other (n=2). All samples were obtained from International Genomic HCC Consortium with IRB approval. After heat-induced antigen retrieval (10 mM sodium citrate buffer (pH 6.0) or Universal HIER antigen retrieval reagent (ab208572) for 15 min (3×5 min)), the reaction was quenched using hydrogen-peroxide 3%, samples were washed with PBS, and incubated with anti-CD8 (Cell Signaling, Danvers, MA) or anti-PD-1 (NAT105, ab52587). DAB (3,3′-diaminobenzidine) was used as a detection system (EnVision+ System-HRP, Dako). PD-1 positive cases were defined considering a) median positivity by immunohistochemistry and b) using a cutoff of >1% of PD-1 positive lymphocytes among all lymphocytes present in each slide. Analysis of human samples from the Department of Pathology and Molecular Pathology, University Hospital Zurich, was approved by the local ethics committee (′Kantonale Ethikkommission Zürich′, KEK—ZH-Nr. 2013-0382 and BASEC-Nr. PB_2018-00252).

Search Strategy, Selection Criteria, and Meta-Analysis of Phase III Clinical Trials

The literature search was done through MEDLINE on PubMed, Cochrane Library, Web of Science, and clinicaltrials.gov, using the following searches: “checkpoint inhibitors”, “HCC”, “phase III”, between January 2010 and January 2020, and complemented by hand searches of conference abstracts/presentations. Single-center, non-controlled trials, studies with insufficient data to extract hazard ratios (HR), 95% confidence intervals, or trials including disease entities other than HCC were excluded. As conference abstracts were not excluded, quality assessment of the included studies was not performed. Three studies fulfilled the criteria and were included in the quantitative synthesis. The primary outcome of the meta-analysis was OS, defined as the time from randomization to death. HRs and CIs related to OS were extracted from the papers/conference presentations. Pooled HRs were calculated using the random-effects model (Der Simonian and Laird), and the generic inverse variance was used for calculating weights64. To evaluate heterogeneity among studies, Cochran's Q test and 12 index were used. A p-value <0.10 in the Q-test was considered to indicate substantial heterogeneity. 12 was interpreted as suggested in the literature: 0% to 40% might not represent significant heterogeneity; 30% to 60% may represent moderate heterogeneity, 50% to 90% may represent substantial heterogeneity, 75% to 100% represents considerable heterogeneity. All statistical pooled analyses were performed using the RevMan 5.3 software.

A Cohort of Patients with HCC Treated with PD-(L)1-Targeted Immunotherapy

The retrospective analysis was approved by local Ethics Committees. Data from this cohort were published previously. Patients with liver cirrhosis and advanced-stage HCC treated with PD-(L)1-targeted immune checkpoint blockers from 12 centers in Austria, Germany, Italy and Switzerland were included. The Chi-square test or Fisher's exact test were used to comparing nominal data. OS was defined as the time from the start of checkpoint inhibitor treatment until death. Patients who were still alive were censored at the date of the last contact. Survival curves were calculated by the Kaplan-Meier method and compared by using the logrank test. Multivariable analysis was performed by a Cox regression model. Statistical analyses were performed using IBM SPSS Statistics version 25 (SPSS Inc., Chicago, IL).

A Validation Cohort of Patients with Hepatocellular Carcinoma Treated with PD-1-Targeted Immune Checkpoint Blockers

A multi-institutional dataset inclusive of 427 HCC patients treated with ICI between 2017 and 2019 in 11 tertiary-care referral centers specialized in the treatment of HCC was analyzed. Clinical outcomes of this patient cohort have been reported elsewhere. Inclusion criteria were: 1) Diagnosis of HCC made by histopathology or imaging criteria according to American Association for the Study of Liver Disease and European Association for the Study of the Liver guidelines; 2) Systemic therapy with ICI for HCC not amenable to curative or loco-regional therapy following local multidisciplinary tumor board review; 3) Measurable disease according to RECIST v1.1 criteria at ICI commencement. From the main study repository, 118 patients with advanced-stage HCC were selected, Child-Pugh A liver functional reserve, and documented radiologic or clinical diagnosis of cirrhosis recruited across the United States (n=85), Europe (n=7), Taiwan (n=14), and Japan (n=12). Ethical approval to conduct this study was granted by the Imperial College Tissue Bank (Reference Number R16008).

Statistical Analyses

Data was collected in Microsoft Excel. Mouse data are presented as the mean±SEM. Pilot experiments and previously published results were used to estimate the sample size, such that appropriate statistical tests could yield significant results. Statistical analysis was performed using GraphPad Prism software version 7.03 (GraphPad Software). Exact p-values lower than p<0.1 are reported and specific tests are indicated in the legends.

### Example 2: Hepatic or Peripheral Blood Derived CD8+ PD-1+ T-Cells Increase During NASH Progression in Mice

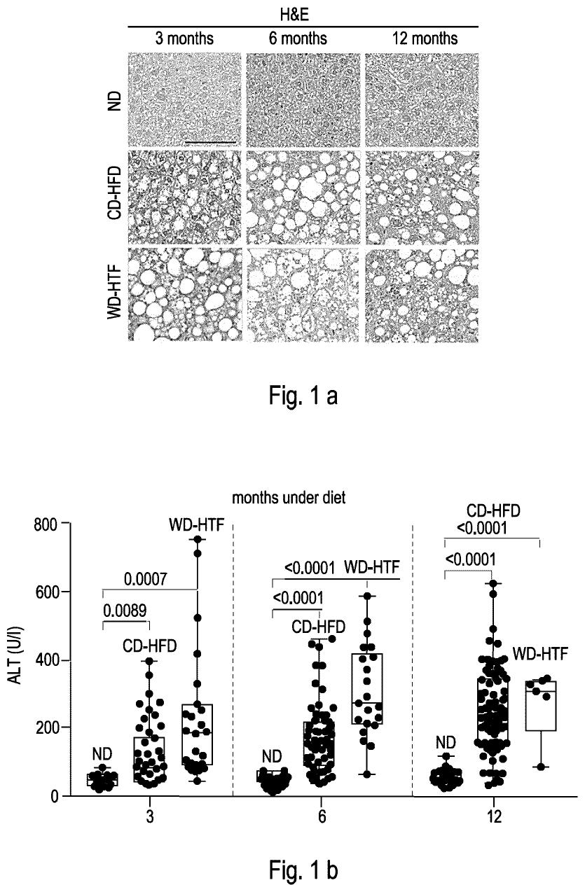

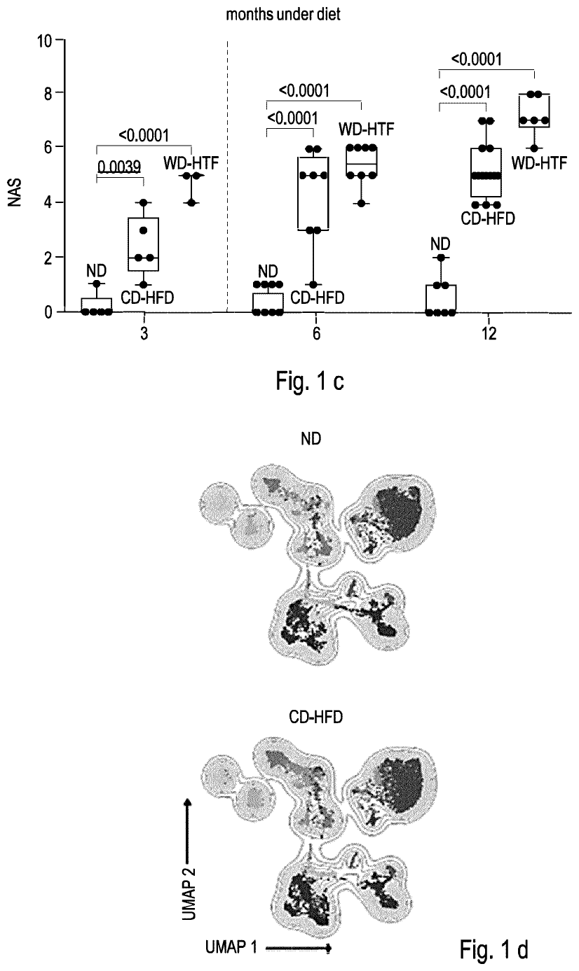

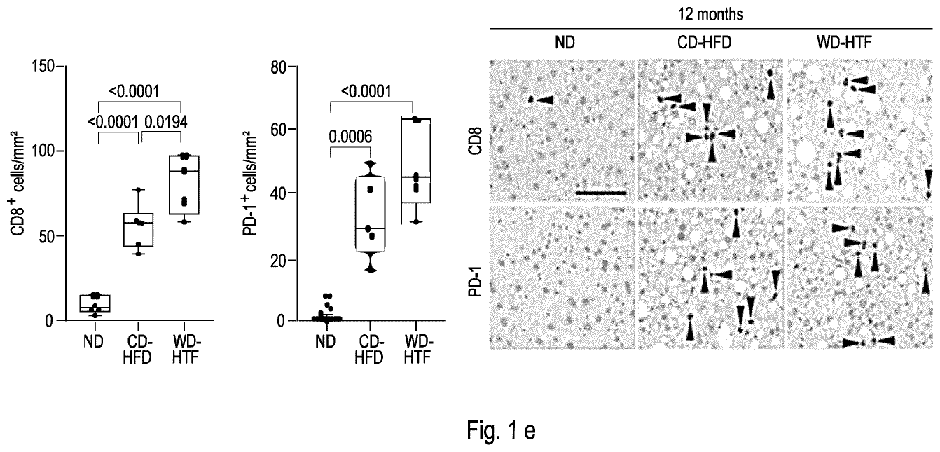

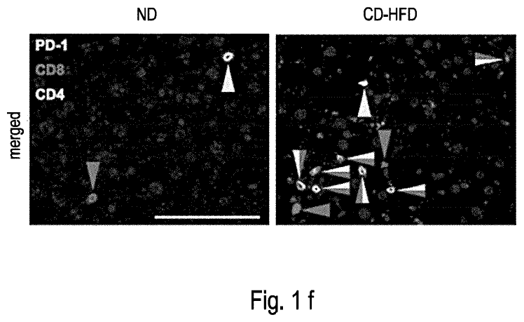

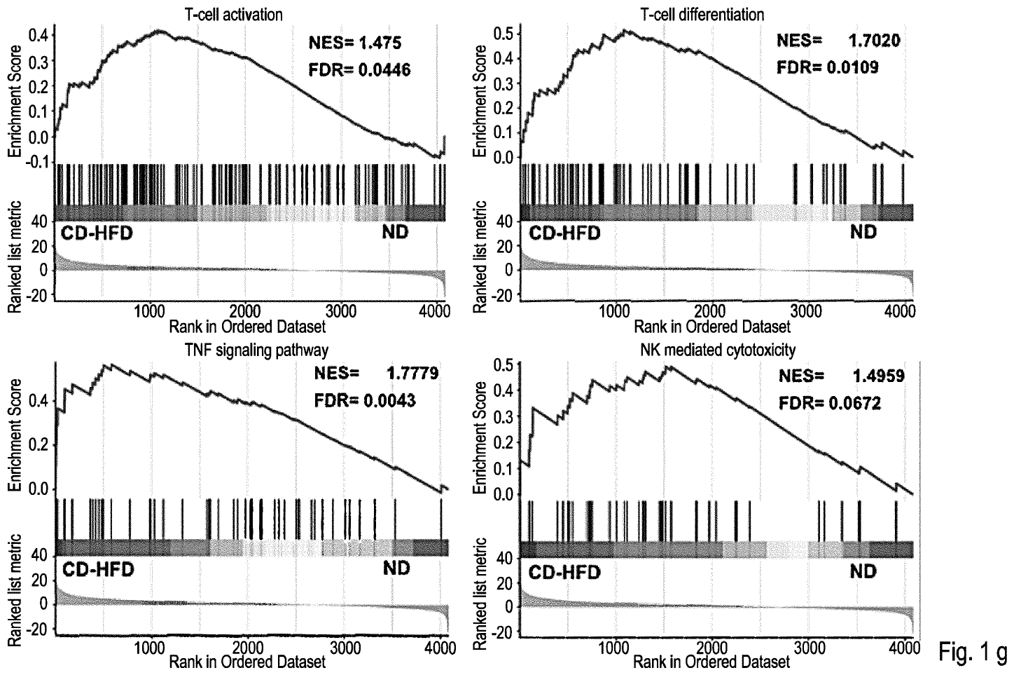

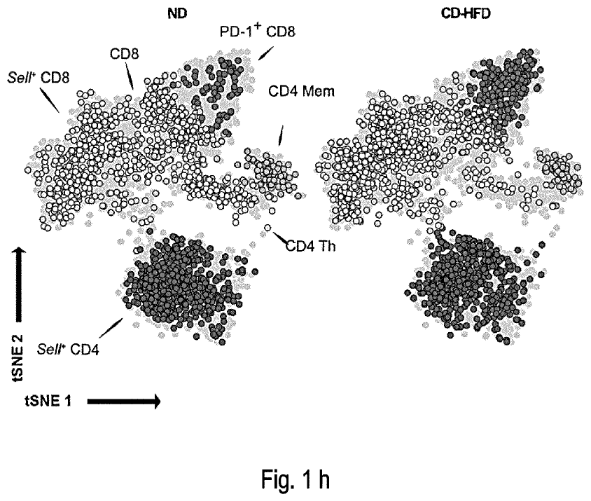

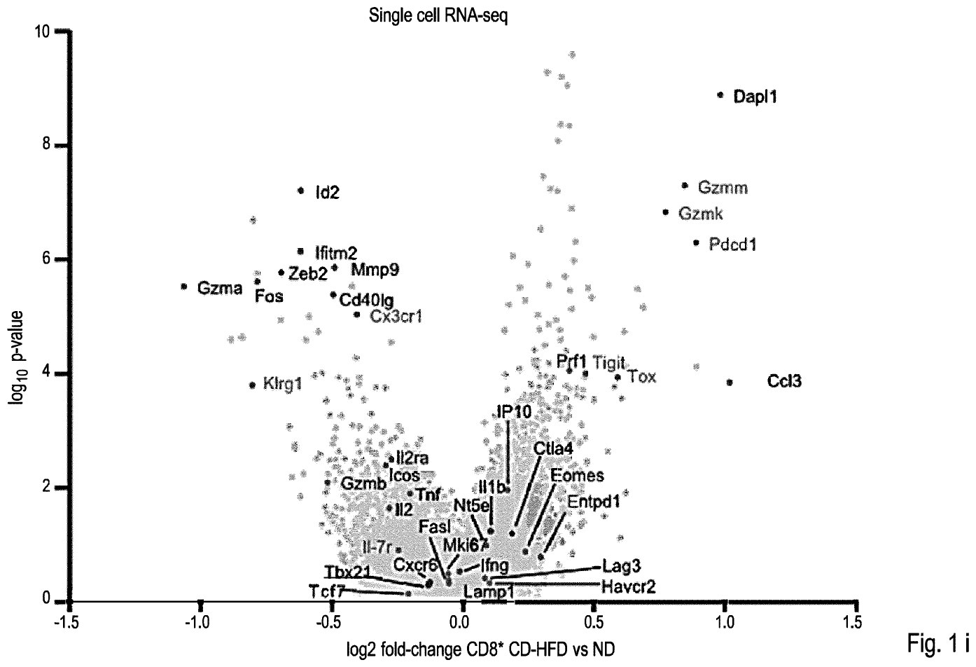

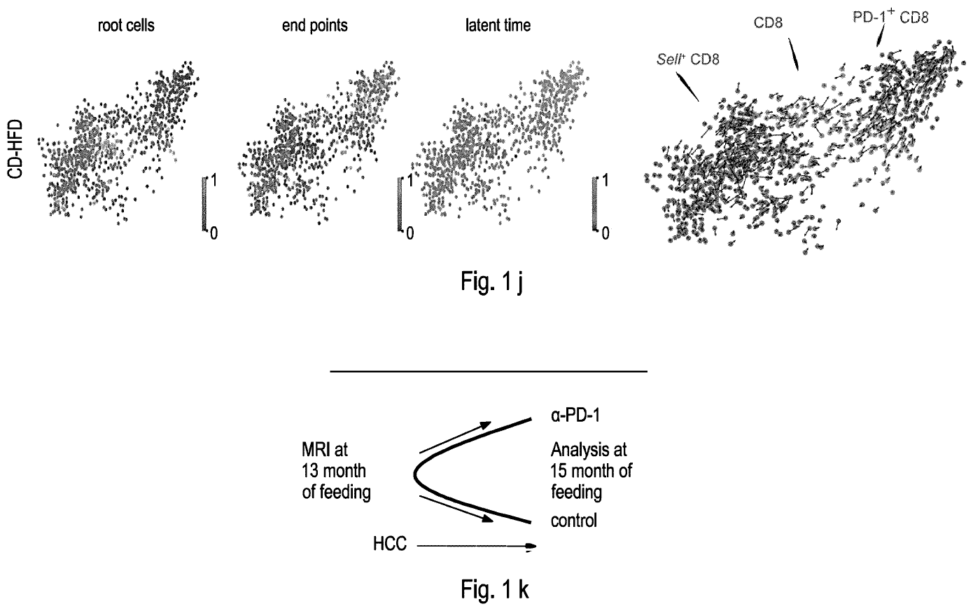

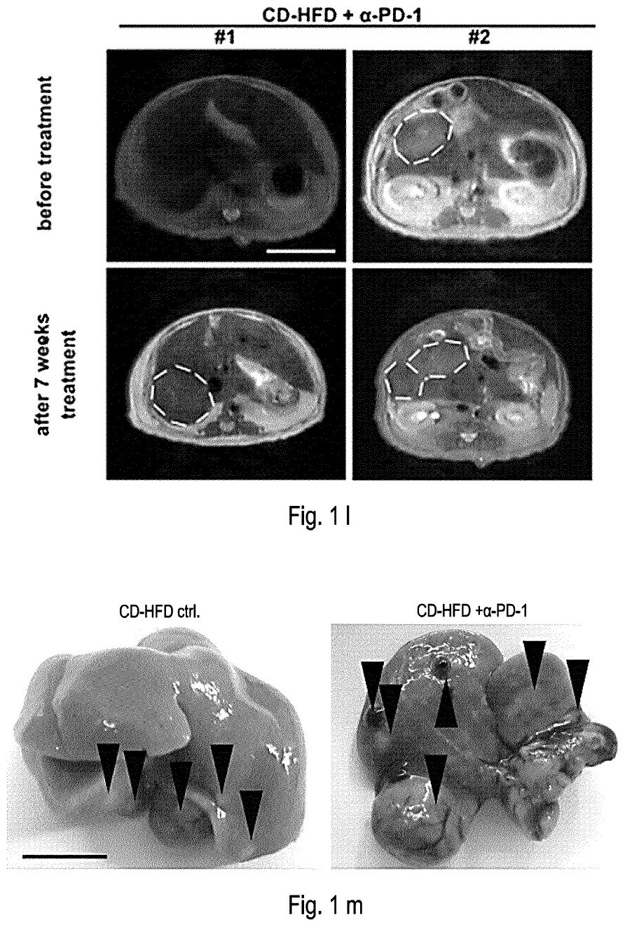

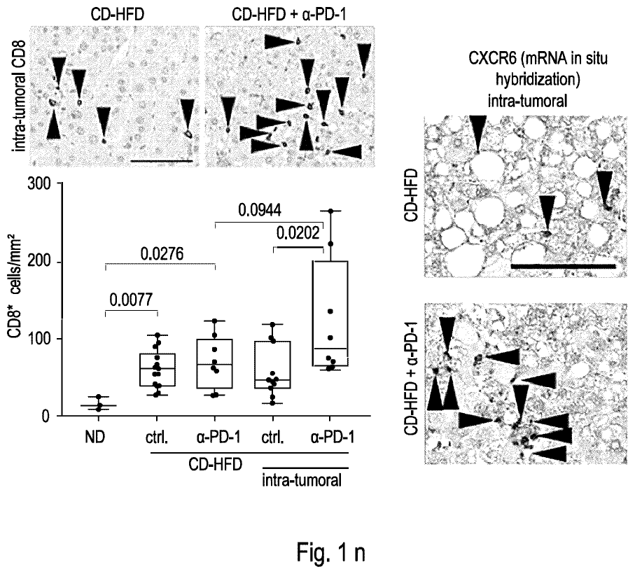

To investigate hepatic or peripheral blood derived immune-cells in NASH, mice were fed with diets, which caused liver damage and NASH in a progressive manner over 3-12 months (FIG. 1a-c), accompanied by increased frequency of hepatic or peripheral blood derived activated CD8+ T-cells expressing CD69/CD44 and PD-1 (Extended Data 1a-d). Single-cell mapping of hepatic or peripheral blood derived leukocytes showed altered immune-cell compositions in NASH (FIG. 1d; Extended Data 1e,f) with strongly increased numbers of CD8+ PD-1+ but not TCRγδ T-cells (FIG. 1e,f). Similarly, elevated CD8+ and PD-1+ cells were found in a genetic NASH mouse model. mRNA in situ hybridization and immunohistochemistry (IHC) revealed increasing PD-L1-expression correlating with NASH-severity in hepatocytes and non-parenchymal cells. Mass spectrometric-based characterization of hepatic or peripheral blood derived CD8+PD-1+ T-cells from NASH-affected livers indicated pathway-enrichment for ongoing T-cell activation and differentiation, TNF-signaling, and NK cell-like cytotoxicity (FIG. 1g). Single-cell RNA sequencing (scRNA-seq) of TCRβ+ cells from NASH livers showed cytotoxicity and effector-function related profiles in CD8+ T-cells (e.g. GzmK/M) and inflammation-markers (e.g. Cc13) with elevated exhaustion traits (e.g. Pdcd1, Tox) (FIG. 1h,i). RNA-velocity analyses demonstrated enhanced transcriptional activity and differentiation starting from SELL-expressing CD8+ to CD8+PD-1+ T-cells (FIG. 1j), indicating a local differentiation process. These data demonstrated increased hepatic or peripheral blood derived abundance of CD8+PD-1+ T-cells in NASH with features of exhaustion and effector function.

Based on the high numbers of hepatic or peripheral blood derived T-cells in NASH, it was asked whether anti-PD-1-targeted immunotherapy serves as an efficient therapy for NASH-HCC. In 30% of C57BL/6 mice fed a CD-HFD for 13-months liver tumors developed, displaying similar load of genetic alterations as human NAFLD/NASH-HCC. Identified by magnetic resonance imaging, NASH-mice bearing HCCs were allocated to anti-PD-1 immunotherapy or a control-arm (FIG. 1k-m). None of the pre-existing liver tumors regressed in response to anti-PD-1 therapy (FIG. 11,m). Rather, more pronounced fibrosis, unchanged liver damage and a slightly increased liver-cancer incidence but no change in tumor-load/-size was observed after anti-PD-1 treatment (FIG. 1k). In anti-PD-1 treated mice, higher numbers of CD8+ and PD1+ T-cells were found in liver-tumor tissue and high levels of CXCR6-, TNF-mRNA expressing cells (FIG. 1n,o). Consistently, no regression of NASH-induced liver tumors was found upon anti-PD-L1 immunotherapy. In contrast, distinct liver-cancer models in non-NASH mice (with or without concomitant damage) reacted to PD-1 immunotherapy with tumor-regression, suggesting that lack of response to immunotherapy was specifically associated with NASH-HCC. Thus, NASH precluded efficient anti-tumor surveillance in the context of HCC-immunotherapy. Similarly, immunotherapy impairment was described in secondary liver cancer in NASH.

### Example 3: CD8+ T-Cells Promote HCC in NASH

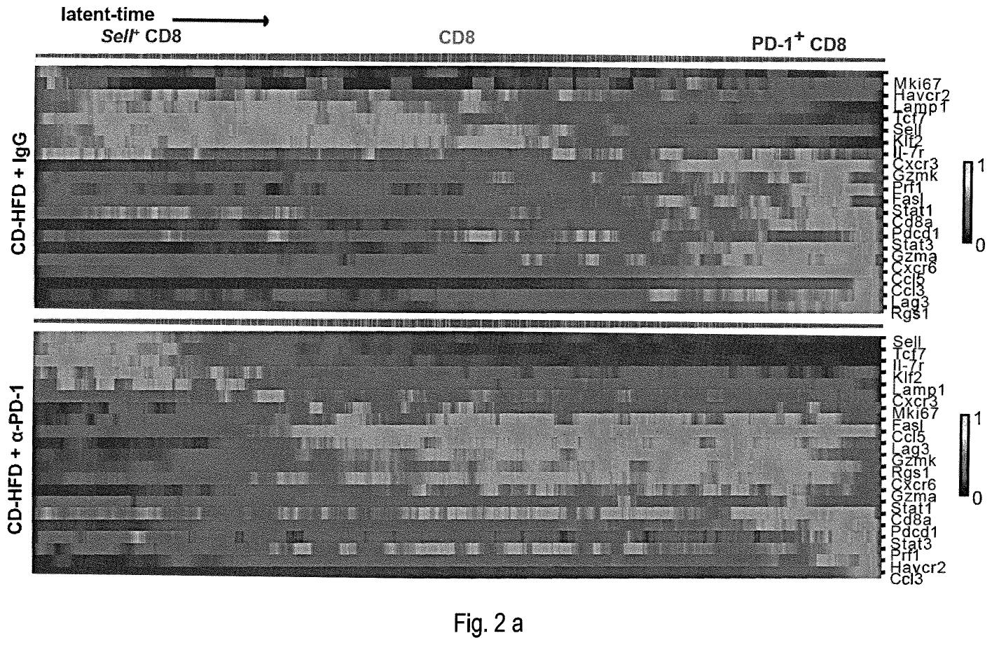

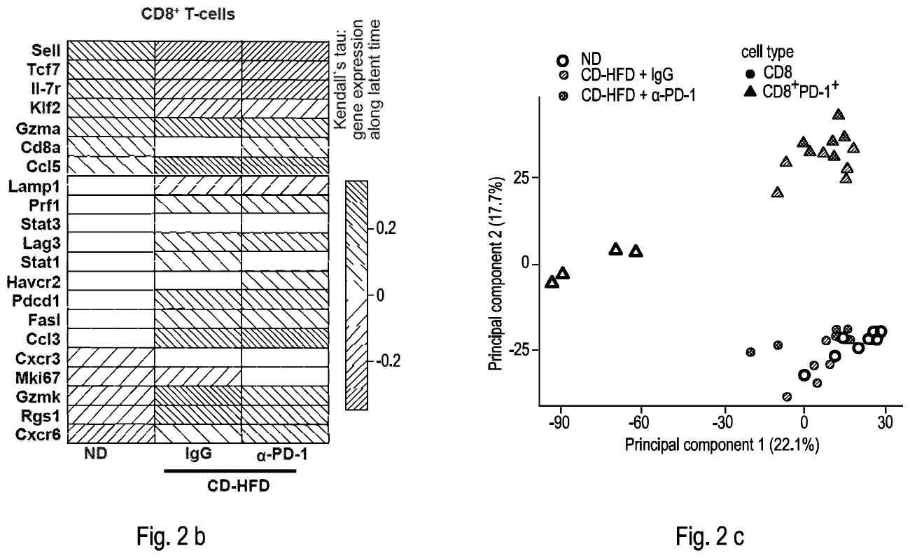

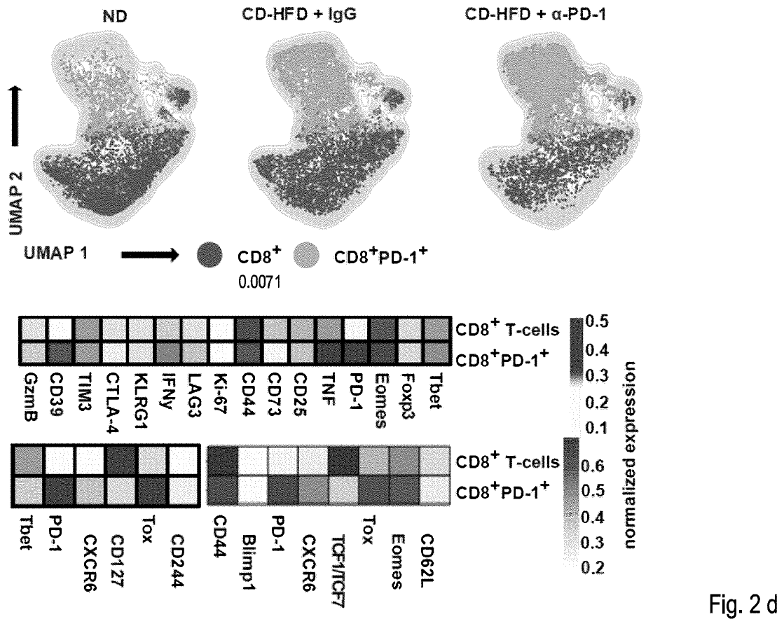

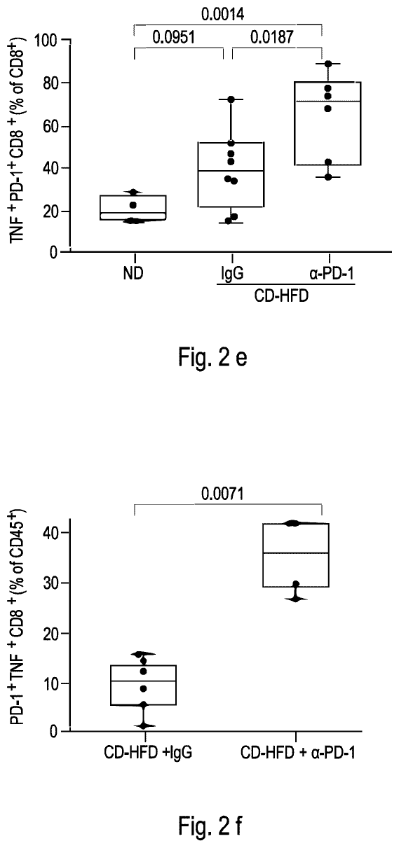

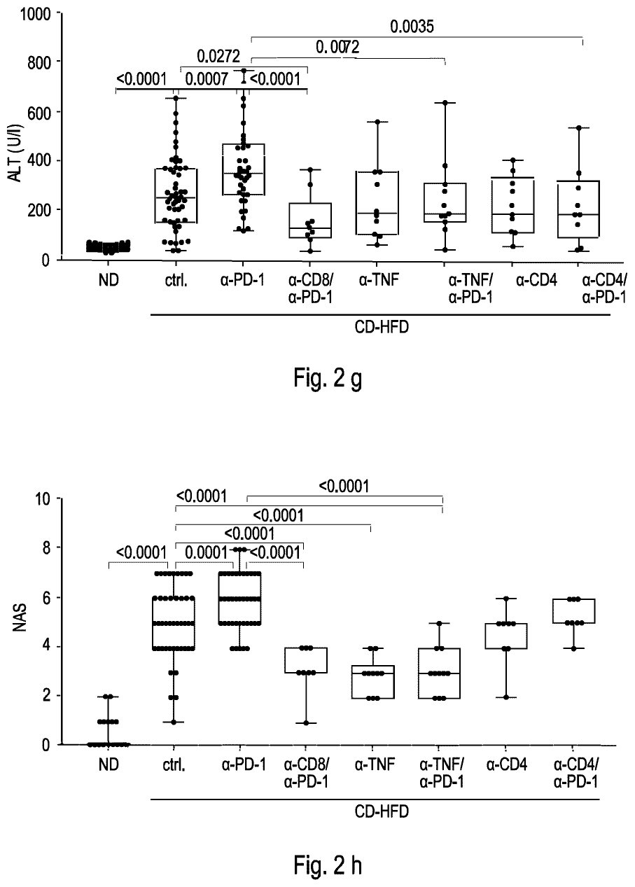

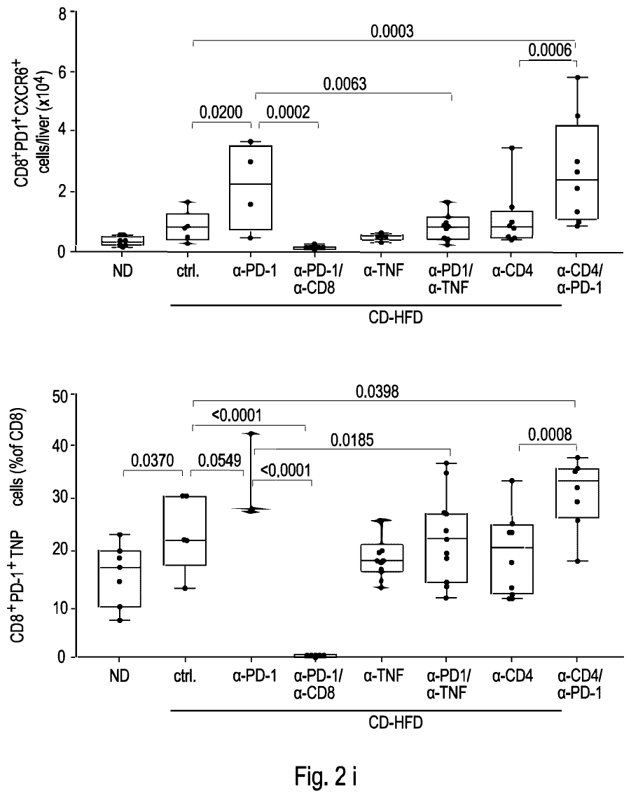

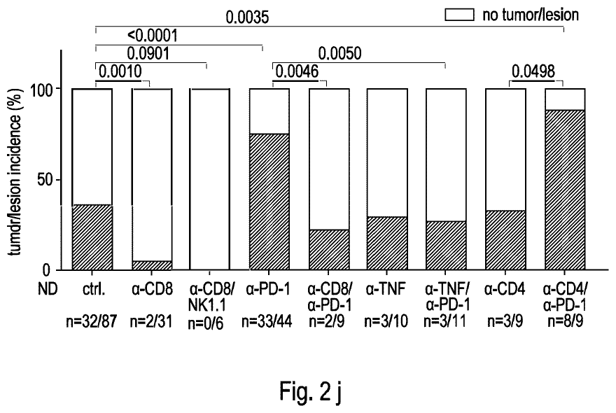

As PD-1+CD8+ T-cells failed to execute effective immune-surveillance but rather showed tissue-damaging potential, we reasoned that CD8+ T-cells might be involved in promoting NASH-HCC and depleted CD8+ T-cells in a preventive setting in mice with NASH, still lacking liver cancer (CD-HFD fed for 10 months). CD8+ T-cell depletion significantly reduced liver damage and decreased HCC incidence [control (vehicle-treated and untreated) n=32/87 (37%) vs. CD8 depletion n=2/31 (6%)] (FIG. 2m). Similar results were obtained after co-depletion of CD8+ and NK1.1+ cells (FIG. 2m). This suggests that liver CD8+ T-cells in NASH not only lack immune-surveillance function in NASH but rather promote HCC. Next, the effect of anti-PD-1 therapy on HCC-development in NASH was investigated. Anti-PD-1 immunotherapy aggravated liver damage and augmented numbers of hepatic or peripheral blood derived CD8+PD-1+ T-cells, whereas only minor changes in liver CD4+PD-1+ T-cells and other immune-cell populations were found. Anti-PD1 immunotherapy caused a dramatic increase in liver-cancer incidence, independent from changes in liver fibrosis (FIG. 2m). This is substantiated by early-onset and increased liver-cancer incidence in PD-1−/− mice already after CD-HFD for 6 months, accompanied by worsened liver damage and increased numbers of activated hepatic or peripheral blood derived CD8+ T-cells with elevated cytokine expression (IFNγ, TNF). In summary, CD8+PD1+ T-cells triggered NASH-HCC transitionc—most likely due to impaired tumor-surveillance and enhanced T-cell mediated tissue damage (see also Dudek et al., 2020). Despite a strong increase of CD8+PD1+ T-cells within tumor tissue, therapeutic PD-1- or PD-L1-related immunotherapy failed to cause tumor-regression in NASH-HCC.

An immune-mediated cancer field (ICF) gene-expression signature associated with human HCC development was applied to understand tumor-driving mechanisms of anti-PD-1 immunotherapy. Preventive anti-PD-1 treatment was strongly associated with the pro-tumorigenic-ICF signature (e.g. Ifnγ, Tnf, Stat3, Stat5, Tgfβ, Kras), capturing traits of T-cell exhaustion, pro-carcinogenic signaling, and mediators of immune-tolerance and inhibition. CD8+ T-cell depletion presented significant downregulation of the high-infiltrate ICF signature and diminished TNF in non-parenchymal cells. GSEA, mRNA in situ hybridization, and histology of tumors developed in NASH mice treated prophylactically with anti-PD1 corroborated these data, finding increased CD8+ T-cell abundance, enrichment for inflammation-related signaling, apoptosis, and TGFβ-signaling. Anti-PD-1 treatment led to increased expression of p62, known to drive hepatocarcinogenesis. Array comparative genomic hybridization indicated no significant differences in chromosomal deletions or amplifications between tumors of anti-PD-1-treated mice or controls. In summary, hepatic or peripheral blood derived CD8+PD-1+ T-cells did no cause tumor regression during NASH but were rather linked to HCC-development, which was even enhanced by anti-PD-1 immunotherapy.

To elucidate the tumor-promoting abilities of CD8+PD-1+ T-cells in NASH after anti-PD-1 treatment, the hepatic or peripheral blood derived T-cell compartment was analyzed for correlation with inflammation and hepatocarcinogenesis. Comparison of CD8+PD-1+ to CD8+ T-cells by scRNA-Seq identified co-expression of genes associated with effector-function (e.g. increased GzmA/B/K, Prf1, Cc13/4/5, reduced SELL, Klf2), exhaustion—(e.g. Pdcd1, Tigit, Tox, reduced Il-7r, Tcf7) and tissue residency (e.g. Cxcr6, Mki-67low) (FIG. 2a-b). Importantly, there was no difference in the transcriptome profile of hepatic or peripheral blood derived CD8+PD-1+ T-cells in NASH-mice after anti-PD-1 (FIG. 2c), indicating that T-cell numbers rather than their functional properties were changed. RNA-velocity-blot analyses corroborated these data (FIG. 2d). Similar patterns of markers (e.g. IL-7r, SELL, Tcf7, Cc15, Cc13, Pdcd1, Cxcr6, FasL, Rgs1) correlated with the latent-time and overall transcriptional activity in NASH mice receiving control IgG or anti-PD-1 (FIG. 2d,e). Mass spectrometry-based analyses of CD8+ or CD8+PD-1+ T-cells isolated from NASH livers confirmed that no phenotypic changes in T-cells occurred after anti-PD-1 treatment (FIG. 2f).

To further characterize the transcriptome profile of PD-1+CD8+ T-cells, UMAP analysis of high-parametric flow-cytometry data was performed dissecting CD8+PD-1+ and CD8+PD-1-subsets (FIG. 2g). This revealed CD8+PD-1+ cell co-expression of high levels of effector—(e.g. GzmB, IFNγ, TNF) and exhaustion-markers (e.g. Eomes, PD-1, Ki-67low). In particular, CD8+ PD-1+ TNF+ cells were more abundant upon anti-PD-1 treatment (FIG. 2h). Convolutional neural network analysis and manual gating validated this result (FIG. 2i). CD8+PD-1+ T-cells were non-proliferative in anti-PD-1-treated NASH mice, supported by in vitro experiments where anti-PD-1 treatment led to an increase of T-cell numbers in the absence of proliferation. Notably, Foxo1 levels of CD8+PD-1+ T-cells were reduced in NASH, indicative of an enhanced tissue-residency phenotype, potentially combined with boosted effector-function, indicated by higher calcium levels in CD8+PD-1+ T-cells. ScRNA-Seq analysis further revealed tissue residency signature of CD8+PD-1+ T-cells in NASH (FIG. 2b). Thus, upon anti-PD1 immunotherapy in NASH, CD8+PD-1+ T-cells accumulated to high numbers in liver, revealing a resident-like T-cell character with increased co-expression of CD44, CXCR6, EOMES, TOX, CD244low, but lacking expression of TCF1/TCF7, CD62L, Tbet, and CD127. Consistent with previous results, the CD4+PD-1+ T-cell compartment was altered. In summary, anti-PD1 immunotherapy increased the abundance of CD8+PD1+ T-cells with a residency signature in liver.

To investigate the mechanisms driving increased NASH-HCC transition in the preventive anti-PD-1 treatment-setting, NASH-affected mice received combinatorial treatments. Anti-CD8/anti-PD-1 or anti-TNF/anti-PD-1 antibody treatment both ameliorated liver damage, liver pathology, and liver inflammation compared to anti-PD-1 treatment alone (FIG. 2j,k). Both combinatorial treatments resulted in decreased liver-cancer incidence compared to anti-PD-1 treatment alone (FIG. 21,m). In contrast, anti-CD4/anti-PD-1 treatment did not reduce liver cancer incidence, NAFLD-score, amount of TNF-expressing hepatic or peripheral blood derived CD8+ or CD8+PD1+CXCR5+ T-cells (FIG. 2j-m). However, a reduction in tumor number per liver and tumor size was observed, suggesting that depletion of CD4+ T-cells or regulatory T-cells might contribute to tumor control. Rather, we found a direct correlation of tumor incidence with anti-PD-1 treatment, ALT, NAS, numbers of hepatic or peripheral blood derived CD8+PD-1+ T-cells, and TNF-expression. Together, these data suggested that CD8+PD1+ T-cells lacked immune-surveillance and had tissue-damaging functions (see also Dudek et al., 2020), which was increased by anti-PD1 treatment and might contribute to the unfavorable effect of anti-PD1 treatment on HCC development in NASH.

In the following Table 1 and Table 2, genes characterizing the hepatic or peripheral blood derived, auto-aggressive CD8+PD-1+ T-cells population are summarized:

TABLE 1
Gene signature of hepatic auto-aggressive
CD8+ PD-1+ T-cells, hepatic resident or
peripheral blood derived

| upregulated compared to | downregulated compared |
|---|---|
| CD8+ T cells | to CD8+ T cells |
| TOX | KLF2 |
| CXCR6 | IL-7R |
| TNFα | TCF7 |
| LAG3 | SELL |
| Granzyme B | Foxo1 |
| TIGIT | CD127 |
| Granzyme A | Tbet |
| Granzyme K | CD62L |
| Prf1 | |
| CCL3 | |
| CCL4 | |
| CCL5 | |
| Pdcd1 | |
| Mki-67low | |
| IFNγ | |
| Eomes | |
| CD44 | |
| CD244low | |

TABLE 2
Important genes of the signature characterizing
the hepatic autoagressive CD8+ PD-1+
T-cells, hepatic resident or peripheral blood derived

| upregulated compared to | downregulated compared |
|---|---|
| CD8+ T cells | to CD8+ T cells |
| TOX | KLF2 |
| CXCR6 | IL-7R |
| TNFα | TCF7 |
| LAG3 | Foxo1 |
| Granzyme B | SELL |
| TIGIT | |

### Example 4: Augmented Liver Resident-Like CD8+ PD1+ T-Cells in NASH Patients

To explore whether similar changes in liver immune-cell characteristics were observed in human NASH, CD8+ T-cells from healthy or NAFLD/NASH-affected livers were investigated. In three independent cohorts of NASH patients, we found enrichment of hepatic or peripheral blood derived CD8+PD-1+ T-cells with a residency phenotype by flow cytometry and CYTOF. Hepatic or peripheral blood derived CD8+PD-1+ T-cell numbers directly correlated with body-mass index and liver damage. To explore similarities between mouse and human T-cells from NASH livers, liver CD8+PD-1+ T-cells from NAFLD/NASH patients were analyzed by scRNAseq, which identified a gene expression signature also found in liver T-cells from NASH mice (e.g. PDCD1, GZMB, TOX, CXCR6, RGS1, SELL). Differentially expressed genes were directly correlated between patient- and mouse-derived hepatic or peripheral CD8+PD-1+ T-cells. Velocity-blot analyses revealed CD8+ T-cells expressing TCF7, SELL, IL-7R as root-cells, and CD8+PD-1+ T-cells, indicating a local developmental trajectory of CD8+ T-cells into CD8+PD-1+ T-cells. Amount of gene expression and velocity magnitude, indicative of transcriptional activity, was increased in mouse and human NASH CD8+PD-1+ T-cells. Marker expression (e.g. IL-7R, SELL, TCF7, CCL5, CCL3, PDCD1, CXCR6, RGS1, KLF2) along the latent-time in NAFLD/NASH patients was different compared to control, and correlated with CD8+ T-cell expression patterns of NASH mice. Thus, scRNAseq analysis demonstrated a resident-like liver CD8+PD-1+ T-cell population in NAFLD/NASH patients that shared gene expression patterns with hepatic or peripheral blood derived CD8+PD-1+ T-cells from NASH mice.

In the following Table 3, genes are listed the expression of which changes over time and which are indicative for root CD8+ T cells that become hepatic auto-aggressive CD8+PD-1+ T-cells, hepatic resident or peripheral blood derived.

TABLE 3

| upregulated over time | downregulated over time |
|---|---|
| TOX | KLF2 |
| CXCR6 | IL-7R |
| TNFα | TCF7 |
| LAG3 | Foxo1 |
| Granzyme B | SELL |
| TIGIT | |

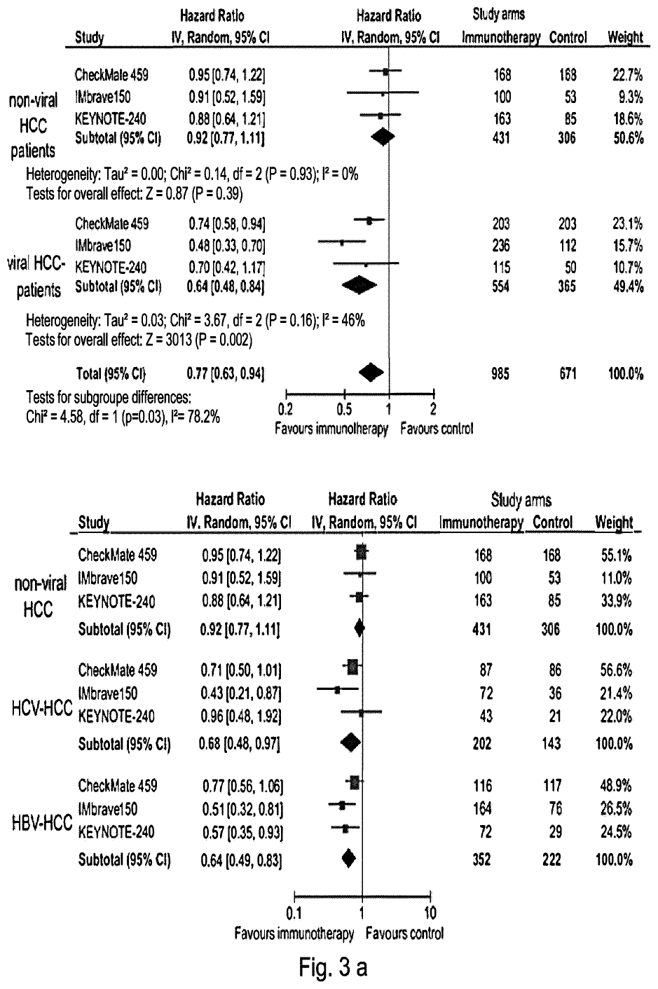

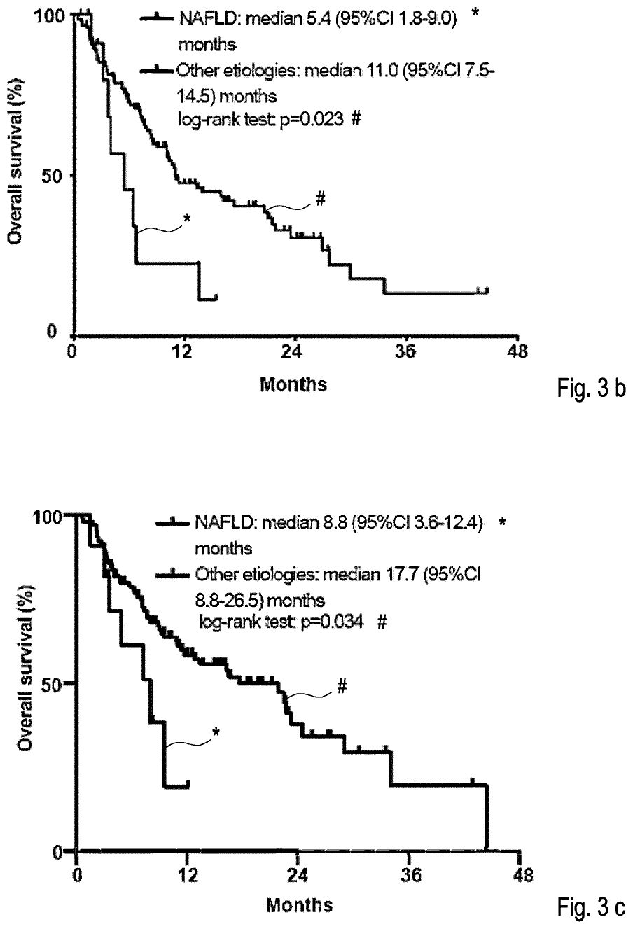

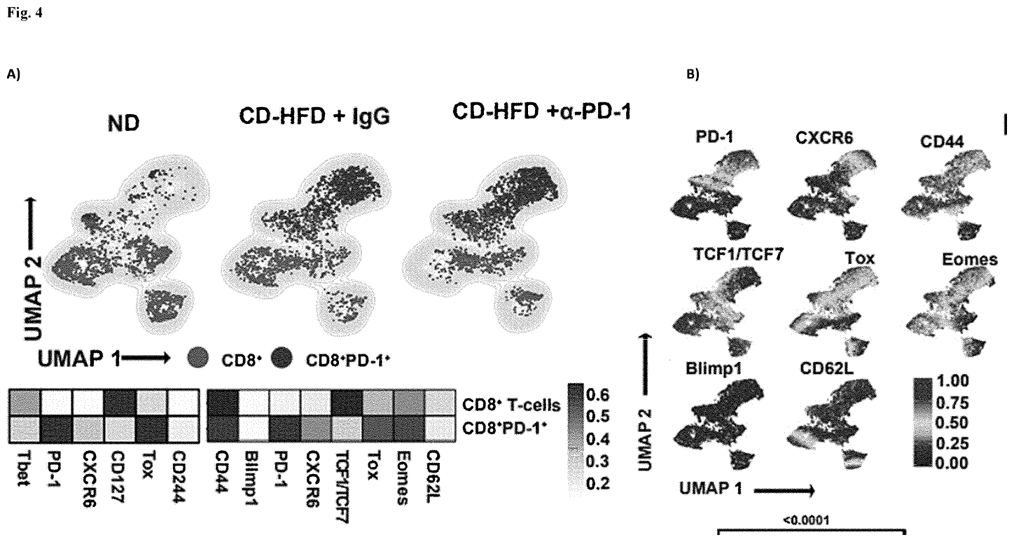

Different stages of NASH-severity are considered to herald liver-cancer development. Indeed, different stages of fibrosis (F0-F4) in NASH directly correlated with expression of Pdcd1, CCL2, IP10, TNF, and degree of fibrosis directly correlated with the amount of CD4+, PD-1+, and CD8+ T-cells (FIG. 3a-c). Moreover, PD-1+ cells were absent in healthy livers but increased in NASH or in NASH-HCC, which did not differ in the underlying level of fibrosis. Species-specific effects such as lack of cirrhosis or burnt-out NASH, a condition found in some NASH-HCC patients, and its possible influence on immunotherapy, may render translation from preclinical NASH models to human NASH difficult. However, in tumor tissue from patients with NASH-induced HCC—treated with anti-PD-1 therapy—increased numbers of intra-tumoral PD-1+ cells were found compared to patients with HCC in viral hepatitis. Thus, a shared gene-expression profile and increased abundance of unconventionally activated hepatic or peripheral blood derived CD8+ PD-1+ T-cells were found in human NASH tissue.

### Example 5: Lack of Response to Immunotherapy in NASH-HCC Patients

To explore the concept of disrupted immune-surveillance in NASH after anti-PD-1/anti-PD-L1 treatment, a meta-analysis for three large randomized controlled phase III studies assessing immunotherapies in patients with advanced HCC was conducted (CheckMate-4591; IMbrave1505; KEYNOTE-24010). While immunotherapy improved survival in the overall population (HR 0.77; 95% CI 0.63-0.94) it was superior to the control arm in HBV (n=574; p=0.0008) and HCV-related HCC patients (n=345; p=0.04), but not in non-viral HCCs (n=737; p=0.39) (FIG. 3e). Patients with viral etiology (HBV and HCV infection) of liver damage and HCC showed a benefit from checkpoint inhibition [HR: 0.64; 95% CI 0.48-0.94] whereas patients with non-viral etiology-HCC did not ([HR: 0.92; 95% CI 0.77-1.11]; p of interaction=0.03 (FIG. 3e)). Subgroup analysis of first-line treatment compared to a control arm treated with sorafenib (n=1243) confirmed that immunotherapy was superior in HBV-related (n=473; p=0.03) and HCV-related HCC patients (n=281; p=0.03), but not in non-viral HCC (n=489; p=0.62). It is acknowledge that results were derived from meta-analysis of trials including different lines of treatment and with heterogeneous nature of liver damage that did not differentiate between alcoholic liver disease and NAFLD/NASH. Nevertheless, results of this meta-analysis supported the notion that stratification of patients according to etiology of liver damage and ensuing HCC identified those patients with a favorable response to therapy.

To specifically characterize the effect of anti-PD-(L)1 immunotherapy with respect to underlying liver disease, a cohort of 130 HCC patients was investigated (NAFLD patients n=13, patients with other etiologies n=117). NAFLD was associated with shortened overall survival after immunotherapy (5.4 (95% CI, 1.8-9.0) months vs. 11.0 (95% CI, 7.5-14.5) months (p=0.023)), although NAFLD patients had less frequent macrovascular tumor-invasion (23% vs 49%), and immunotherapy was more often used as first-line therapy (46% vs. 23%) (FIG. 3f). After corrector for potentially confounding factors relevant for prognosis including severity of liver damage, macrovascular tumor-invasion, extrahepatic metastases, performance status, and alpha-fetoprotein (AFP), NAFLD remained independently associated with shortened survival of HCC patients after anti-PD1-treatment (HR 2.6 (95% CI, 1.2-5.6; p=0.017). This was validated in a further cohort of 118 HCC-patients treated with PD-(L)1-targeted immunotherapy (NAFLD n=11, patients with other etiologies n=107). NAFLD was again associated with reduced survival of HCC patients (median 8.8 months, 95% CI 3.6-12.4) compared to other etiologies of liver damage (median OS 17.7 months 95% CI 8.8-26.5, p=0.034) (FIG. 3g). Given the relatively small number of NAFLD patients in both cohorts, these data need prospective validation. However, collectively these results indicated that patients with underlying NASH did not benefit from checkpoint-inhibition therapy.

Liver cancer develops primarily on the basis of chronic inflammation. The latter can be activated by immunotherapy to induce tumor-regression in a subset of liver cancer patients. However, the identity of responders to immunotherapy for HCC remains elusive. The present data identify a non-viral etiology of liver damage and cancer, i.e. NASH, as a predictor of unfavorable outcome in patients treated with immune-checkpoint inhibitors. Better response to immunotherapy in viral-induced HCC patients compared to non-viral HCC patients might be due to the amount or quality of viral-antigens or a different liver micro-environment, possibly not impairing immune-surveillance. The present results might also have implications for obese patients with NALFD/NASH suffering from cancer at other organ sites (e.g. melanoma, colon carcinoma, breast cancer) and at risk for liver damage and development of liver cancer in response to systemically applied immunotherapy. Overall, a comprehensive mechanistic insight and a rational basis for HCC patient-stratification according to etiology of liver damage and cancer for future trial designs in personalized cancer therapy was provided.

### CITED LITERATURE

Llovet, J. M. et al. Hepatocellular carcinoma. Nat. Rev. Dis. Prim. 7, 6 (2021).
Galle, P. R. et al. EASL Clinical Practice Guidelines: Management of hepatocellular carcinoma. J. Hepatol. 69, 182-236 (2018).
Zhu, A. X. et al. Pembrolizumab in patients with advanced hepatocellular carcinoma previously treated with sorafenib (KEYNOTE-224): a non-randomised, open-label phase 2 trial. Lancet Oncol. 2045, 1-13 (2018).
El-Khoueiry, A. B. et al. Nivolumab in patients with advanced hepatocellular carcinoma (CheckMate 040): an open-label, non-comparative, phase 1/2 dose escalation and expansion trial. Lancet 6736, 1-11 (2017).
Finn, R. S. et al. Atezolizumab plus bevacizumab in unresectable hepatocellular carcinoma. N. Engl. J. Med. 382, 1894-1905 (2020).
Roderburg, C., Wree, A., Demir, M., Schmelzle, M. & Tacke, F. The role of the innate immune system in the development and treatment of hepatocellular carcinoma. Hepatic Oncol. hep-2019-0007 (2020) doi:10.2217/hep-2019-0007.
Duffy, A. G. et al. Tremelimumab in Combination with Ablation in Patients with Advanced Hepatocellular Carcinoma. J. Hepatol. 66, 482-484 (2016).
Sangro, B. et al. A clinical trial of CTLA-4 blockade with tremelimumab in patients with hepatocellular carcinoma and chronic hepatitis C. J. Hepatol. 59, 81-88 (2013).
Anstee, Q. M., Reeves, H. L., Kotsiliti, E., Govaere, 0. & Heikenwalder, M. From NASH to HCC: Current concepts, future challenges. Nat 424 Rev Gastroenterol Hepatol 411-428 (2019).
Wolf, M. J. et al. Metabolic activation of intrahepatic CD8+ T cells and NKT cells causes nonalcoholic steatohepatitis and liver cancer via cross-talk with hepatocytes. Cancer Cell 26, 549-64 (2014).
Ma, C. et al. NAFLD causes selective CD4+ T lymphocyte loss and promotes hepatocarcinogenesis. Nature 531, 253-257 (2016).
Malehmir, M. et al. Platelet GPIbα is a mediator and potential interventional target for NASH and subsequent liver cancer. Nat. Med. 25, 641-655 (2019).
Nakagawa, H. et al. E R Stress Cooperates with Hypernutrition to Trigger TNF Dependent Spontaneous HCC Development. Cancer Cell 26, 331-343 (2014).
Ringelhan, M., Pfister, D., O'Connor, T., Pikarsky, E. & Heikenwalder, M. The immunology of hepatocellular carcinoma. Nat. Immunol. 19, (2018).
Michelotti, G. a, Machado, M. V & Diehl, A. M. NAFLD, NASH and liver cancer. Nat. Rev. Gastroenterol. Hepatol. 10, 656-65 (2013).
Friedman, S. L., Neuschwander-Tetri, B. A., Rinella, M. & Sanyal, A. J. Mechanisms of NAFLD development and therapeutic strategies. Nat. Med. 24, 908-922 (2018).
EP 1 504 126 A2

1. A method for assessing a treatment response associated with immunotherapy in a subject in need thereof comprising the steps of:
(a) determining (i) hepatic auto-aggressive CD8 positive (+) PD-1 positive (+) T cells exhibiting traits of activation and exhaustion or (ii) CD8+ T cell precursors thereof in a sample of a subject in need of immunotherapy; and
(b) assessing the treatment response associated with immunotherapy based on the presence, absence or abundance of (i) said hepatic auto-aggressive CD8+ PD-1+ T-cells exhibiting traits of activation and exhaustion or (ii) said CD8+ T cell precursors thereof.

2. A method for assessing a treatment response associated with immunotherapy in a subject in need thereof comprising the steps of:
(a) determining data indicating the presence, absence or abundance of (i) hepatic auto-aggressive CD8 positive (+) PD-1 positive (+) T cells exhibiting traits of activation and exhaustion or (ii) CD8+ T cell precursors thereof in a data set comprising imaging data of a subject in need of immunotherapy; and
(b) assessing the treatment response associated with immunotherapy based on the presence, absence or abundance of (i) said hepatic auto-aggressive CD8+ PD-1+ T-cells exhibiting traits of activation and exhaustion or (ii) said CD8+ T cell precursors thereof.

3. The method of claim 1, wherein said treatment response is the absence of or an adverse treatment response.

4. The method of claim 3, wherein the presence of (i) said hepatic auto-aggressive CD8+ PD-1+ T-cells exhibiting traits of activation and exhaustion or (ii) said CD8+ T cell precursors thereof is indicative for an absence of or an adverse treatment response associated with immunotherapy.

5. The method of claim 1, wherein said treatment response is a therapeutically effective treatment response.

6. The method of claim 5, wherein the absence of (i) said hepatic auto-aggressive CD8+ PD-1+ T-cells exhibiting traits of activation and exhaustion or (ii) said CD8+ T cell precursors thereof is indicative for a therapeutically effective treatment response associated with immunotherapy.

7. The method of claim 1, wherein said subject suffers or is suspected to suffer from non-alcoholic fatty liver disease (NAFLD) or systemic obesity (metabolic syndrome).

8. The method of claim 7, wherein said treatment response is an adverse hepatic side effect.

9. The method of claim 8, wherein the presence of (i) said hepatic auto-aggressive CD8+ PD-1+ T-cells exhibiting traits of activation and exhaustion or (ii) said CD8+ T cell precursors thereof is indicative for an adverse hepatic side effect associated with immunotherapy.

10. The method of claim 1, wherein said immunotherapy involves PD-1 and/or PD-L1 targeted immunotherapy.

11. The method of claim 1, wherein said hepatic auto-aggressive CD8+ PD-1+ T cells exhibiting traits of activation and exhaustion exhibit an increased expression compared to control CD8+ T cells of at least one biomarker selected from the group consisting of: TOX, CXCR6, TNFα, LAG3, GZMB (Granzyme B) and TIGIT.

12. The method of claim 1, wherein said hepatic auto-aggressive CD8+ PD-1+ T cells exhibiting traits of activation and exhaustion exhibit a reduced expression compared to control CD8+ T cells of at least one biomarker selected from the group consisting of: KLF2, IL-7R, TCF7, Foxo1 and SELL.

13. The method of claim 1, wherein said CD8+ T cell precursors are characterized by at least one biomarker selected from the group consisting of: TCF7, SELL, and IL-7R.

14. The method of claim 13, wherein said CD8+ T cell precursors exhibit a change in expression over time of at least one biomarker selected from the group consisting of: TOX, CXCR6, TNFα, LAG3, GZMB (Granzyme B) TIGIT, KLF2, IL-7R, TCF7, Foxo1 and SELL.

15. The method of claim 14, wherein (i) said change is a decrease in expression over time if said at least one biomarker is selected from the group consisting of KLF2, IL-7R, TCF7, Foxo1 and SELL; and (ii) said change is an increase in expression over time if said at least one biomarker is selected from the group consisting of TOX, CXCR6, TNFα, LAG3, GZMB (Granzyme B) and TIGIT.

16. A method for recommending immunotherapy for a subject comprising assessing the treatment response to immunotherapy for said subject by carrying out the method of 1 and, recommending immunotherapy for said subject if the subject is assessed to have no non-treatment response, no adverse treatment response, a therapeutically effective treatment response and/or no adverse hepatic side effect.
